(19) United States
(12) Patent Application Publication
(10) Pub. No.: US 2024/0390384 A1
(43) Pub. Date: Nov. 28, 2024

(54) METHODS FOR ACUTE AND LONG-TERM TREATMENT OF OPIOID AND OPIOID-LIKE DRUG ADDICTION

(71) Applicant: DemeRx IB, Inc., Miami, FL (US)

(72) Inventor: Lawrence FRIEDHOFF, River Vale, NJ (US)

(21) Appl. No.: 18/396,955

(22) Filed: Dec. 27, 2023

Related U.S. Application Data
(63) Continuation of application No. 17/480,954, filed on Sep. 21, 2021, now abandoned.
Application No. 17/480,954 is a continuation of application No. 15/786,454, filed on Oct. 17, 2017, now abandoned.
Application No. 15/786,454 is a continuation of application No. 14/635,842, filed on Mar. 2, 2015, now abandoned.
(60) Provisional application No. 61/947,397, filed on Mar. 3, 2014.

(51) Int. Cl. A61K 31/55

(52) U.S. Cl. CPC A61K 31/55

(57) ABSTRACT

This invention is directed to a method of treating opioid or opioid-like drug addiction, including acute and post-acute withdrawal symptoms, comprising treating an addicted patient with ibogaine, ibogaine derivative, or pharmaceutically acceptable salt or solvate thereof at a dosage that provides an average serum concentration of about 50 ng/mL to about 850 ng/mL under conditions where the QT interval prolongation does not exceed about 50 milliseconds.

### CROSS-REFERENCE TO RELATED APPLICATIONS

This application claims benefit from U.S. Provisional Application No. 61/947,397, filed Mar. 3, 2014, which is hereby incorporated by reference in its entirety.

### FIELD OF THE INVENTION

This invention is directed to a method of treating addiction to an opioid or opioid-like drug, including acute and post-acute withdrawal symptoms, comprising treating an opioid-addicted patient with ibogaine, ibogaine derivative, or pharmaceutically acceptable salt or solvate thereof at a therapeutic dosage that provides both an average serum concentration of about 50 ng/mL to about 850 ng/mL and a maximum QT interval prolongation of no more than about 50 milliseconds.

### STATE OF THE ART

Substance addiction is a serious public health problem throughout the world. Heroin and other opioids, including prescription painkillers, are widely abused and account for a large percentage of illicit drug use. Opioid use is also linked to approximately 50% of violent crimes in the United States and costs the U.S. economy billions of dollars per year.

Acute withdrawal from drug dependence is characterized by dramatic and traumatic symptoms, including sweating, racing heart, palpitations, muscle tension, tightness in the chest, difficulty breathing, tremor, nausea, vomiting, diarrhea, grand mal seizures, heart attacks, strokes, hallucinations and delirium tremens (DTs). Once acute withdrawal symptoms have subsided, post-acute withdrawal syndrome can last for months or years. Post-acute withdrawal symptoms include fatigue, depression, lack of motivation, and increased pain sensitivity.

Numerous treatments have been developed in attempts to ameliorate acute and post-acute withdrawal symptoms. However, in most cases, treatment of withdrawal requires use of other addictive substances (e.g., morphine or methadone). Treatment also requires that the addict attend a clinic daily for an extended amount of time. Due to the severity and duration of withdrawal symptoms, opioid-addicted patients have a high rate of relapse. There is a significant need for effective, non-addictive treatment for acute and post-acute opioid withdrawal symptoms.

Ibogaine has been used as a botanical preparation from the root bark of iboga tabernathe for over 100 years both as a crude preparation and as semisynthetic ibogaine, which was marketed in France until about 1970. Ibogaine has been used in the treatment of drug addiction, including addiction to opiates and other narcotics (U.S. Pat. No. 4,857,523), alcohol (U.S. Pat. No. 4,857,523), and nicotine (U.S. Pat. No. 5,026,697).

Prior to the embodiments described herein, the therapeutic dosing of ibogaine and its derivatives for treating opioid or opioid-like drug addiction in humans at an acceptable QT interval prolongation has not previously been addressed, especially as it relates to dosing protocols that are effective, as well as safe.

### SUMMARY

While ibogaine has been disclosed for treatment of substance addiction, its use in humans is complicated by the fact that the ranges generally used to treat addiction (e.g., 15 mg/kg to 20 mg/kg) cause hallucinations and may be fatal. Lotsof and Wachtel, Manual for Ibogaine Therapy: Screening, Safety, Monitoring & Aftercare (2d revision, 2003), accessed at www.ibogaine.desk.nl/manual.html; Hoelen, et al. New Engl. J. Med. 360(3), 308 (2009), which is incorporated herein by reference in its entirety for all of its methods, compositions and teachings.

A prolonged QT interval is a marker of potential ventricular tachyarrhythmia which, and can result in death. Serious complications, including ventricular tachyarrhythmia and death, can result from prolongation of the treated patient's QT interval by ibogaine, rendering high doses of ibogaine unacceptable. Heretofore, it was unclear whether a therapeutic dose of ibogaine could be found that resulted in QT interval prolongation within an acceptable range. It is expected that other compounds that share ibogaine's core structure will have a similar prolongation effect on QT interval. See, U.S. Provisional Patent Application No. 61/945,746 filed Feb. 27, 2014 entitled METHOD FOR ACUTE AND LONG-TERM TREATMENT OF DRUG ADDICTION, which application is incorporated by reference in its entirety.

The current invention is predicated on the surprising discovery that treatment of addiction with ibogaine, ibogaine derivative, or pharmaceutically acceptable salt or solvate thereof can be achieved with an acceptable QT interval prolongation when such compounds are administered within a narrow dosage range. Specifically, dosing an addicted patient with from greater than about 1 mg/kg body weight to about 4 mg/kg body weight ibogaine, ibogaine derivative, or pharmaceutically acceptable salt or solvate thereof will provide a therapeutic reduction in withdrawal symptoms and/or an increase in time to resumption of opioid use in opioid-addicted patients without unacceptable prolongation of the patient's QT interval.

In some embodiments, the ibogaine is represented by Formula I:

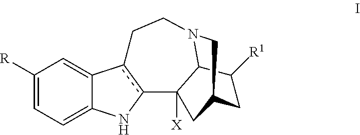

or a pharmaceutically acceptable salt thereof,
wherein
R is hydrogen or C₁-C₃-alkoxy,
R¹ is hydrogen, C₁-C₃-alkyl, C₁-C₃ alkoxy, or CH₂—Y—CH₃ where Y is O or NH, and
X is H, COOH, or COOR², where R² is C₁-C₆ alkyl or (CH₂CH₂O)ⁿCH₃, where n=1 to 3.

In one embodiment, R is methoxy. In one embodiment, R¹ is methoxy. In one embodiment, R¹ is CH₂—Y—CH₃ where Y is O. In one embodiment, R¹ is CH₂—Y—CH₃ where Y is NH. In one embodiment, R² is methyl. In one embodiment, n=1. In a preferred embodiment, R, R¹ and X are all not hydrogen. In one embodiment, when R is methoxy and R¹ is hydrogen, then X is COOH or COOR². In another embodiment, when R is methoxy and R¹ is hydrogen, then X is COOR² where R² is (CH₂CH₂O)CH₃.

Preferably, the dose range that provides both therapeutic results and an acceptable QT interval prolongation of less than 50 milliseconds in opioid and opioid-like drug addicted humans is between about 1 3 mg per kg body weight and no more than about 4 mg per kg body weight and, more preferably between about 1.3 mg per kg body weight and no more than about 3 mg per kg body weight, or any subrange or subvalue within the aforementioned ranges. Opioid-like drugs, including cocaine, ketamine, and methamphetamine, are not opioids but act through the opioid receptors, and thus addiction to these drugs also can be treated with ibogaine, ibogaine derivative, or pharmaceutically acceptable salt or solvate thereof.

In a preferred embodiment, the narrow therapeutic doses ibogaine, ibogaine derivative, or pharmaceutically acceptable salt or solvate thereof unexpectedly do not prolong the QT interval to unacceptable levels in human addicted patients. It is expected that opioid or opioid-like drug addicted patients will be administered therapeutic doses of ibogaine, ibogaine derivative, or pharmaceutically acceptable salt or solvate thereof a clinical setting with cardiac monitoring. In some embodiments, the patient will be pre-screened to evaluate tolerance for prolongation of QT interval, e.g., to determine whether the patient has any pre-existing cardiac conditions which would disqualify them from treatment with ibogaine, ibogaine derivative, or pharmaceutically acceptable salt or solvate thereof.

Some aspects of the current invention are further predicated on the discovery that even lower doses of ibogaine, ibogaine derivative, or pharmaceutically acceptable salt or solvate thereof, for example approximately 80% or less of the therapeutic dose, may be effective for prevention of relapse of opioid (or opioid-like drug) use in an opioid-addicted patient treated to ameliorate their opioid use. That is, a lower dose of the compound can prevent a patient who is no longer physically addicted to opioid from relapsing to opioid use. Without being bound by theory, it is believed that a patient who is no longer physically addicted to opioids or opioid-like drug requires less compound to prevent relapse because the opioid or opioid-like drug does not compete with the compound for receptor binding, and/or because desensitization of one or more receptors in the brain by the opioid or opioid-like drug is reversed when the patient ceases to take the drug. This lower, maintenance dose results in a QT interval prolongation that does not require clinical cardiac monitoring.

In some embodiments, the therapeutic dose of ibogaine, ibogaine derivative, or pharmaceutically acceptable salt or solvate thereof administered to the patient is sufficient to provide an average serum concentration of the compound of about 50 ng/mL to about 850 ng/mL, or any subrange or subvalue there between. In a preferred embodiment, the dose of ibogaine, ibogaine derivative, or pharmaceutically acceptable salt or solvate thereof administered to the patient provides an average serum concentration of about 50 ng/mL to about 400 ng/mL.

In some embodiments, the patient is administered a high (therapeutic) dose of ibogaine, ibogaine derivative, or pharmaceutically acceptable salt or solvate thereof for a period of time to ameliorate the most significant withdraw symptoms, and then is administered a lower (maintenance) dose to prevent relapse to opioid or opioid-like drug use. In some embodiments, the patient is administered a therapeutic dose of ibogaine, ibogaine derivative, or pharmaceutically acceptable salt or solvate thereof for a period of time to ameliorate the most significant withdraw symptoms, and then is administered a decreasing (tapered) amount of ibogaine, ibogaine derivative, or pharmaceutically acceptable salt or solvate thereof over time until the maintenance dose is reached. In some embodiments, a high initial therapeutic dose is administered, followed by administration of a lower therapeutic dose. In some embodiments, the dose of the compound is tapered over time from the high therapeutic dose to a lower therapeutic dose.

In some embodiments, the dose of ibogaine, ibogaine derivative, or pharmaceutically acceptable salt or solvate thereof that provides an average serum concentration of about 50 ng/mL to about 850 ng/mL is administered as a single dose. In some embodiments, the dose of ibogaine, ibogaine derivative, or pharmaceutically acceptable salt or solvate thereof that provides an average serum concentration of about 50 ng/mL to about 850 ng/mL is administered as multiple doses. In some embodiments, the aggregate dose of ibogaine, ibogaine derivative, or pharmaceutically acceptable salt or solvate thereof is from greater than about 1 mg/kg to about 8 mg/kg. In a preferred embodiment, the aggregate dose of ibogaine, ibogaine derivative, or pharmaceutically acceptable salt or solvate thereof is from greater than about 1 mg/kg to about 4 mg/kg. In another preferred embodiment, the aggregate dose of ibogaine, ibogaine derivative, or pharmaceutically acceptable salt or solvate thereof is from greater than about 1 mg/kg to 3 mg/kg.

In some embodiments, the serum concentration is sufficient to inhibit or ameliorate said abuse while maintaining a QT interval of less than 500 milliseconds (ms) during said treatment. In some embodiments, the therapeutic dose of ibogaine, ibogaine derivative, or pharmaceutically acceptable salt or solvate thereof provides prolongation of the QT interval of less than 80 ms. In a preferred embodiment, the maintenance dose of ibogaine, ibogaine derivative, or pharmaceutically acceptable salt or solvate thereof provides prolongation of the QT interval of less than 50 ms. In some embodiments, the maintenance dose or therapeutic dose of ibogaine, ibogaine derivative, or pharmaceutically acceptable salt or solvate thereof provides prolongation of the QT interval of less than 30 ms. In a preferred embodiment, the maintenance dose of ibogaine, ibogaine derivative, or pharmaceutically acceptable salt or solvate thereof provides prolongation of the QT interval of less than 20 ms. In one embodiment, the QT prolongation is equivalent to or less than that observed in patients receiving methadone treatment. In a preferred embodiment, the patient is tested to determine QT interval before treatment with the compound, and if clinician determines that the QT prolongation would be unacceptable risk, therapy will be contraindicated.

### Compounds Administered

In the various method, formulation and kit aspects and embodiments, in one embodiment a compound utilized herein is represented by, or ibogaine as used herein is replaced by, a compound Formula I:

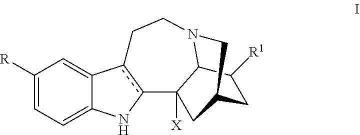

or a pharmaceutically acceptable salt and/or solvate thereof,
wherein
R is H, halo, C₁-C₃ alkyl, substituted C₁-C₃ alkyl, OR¹⁰, NH₂, NHR¹⁰, NR¹⁰R¹¹, NHC(O)R¹⁰, or NR¹⁰C(O)R¹¹;
R¹ is H, C₁-C₃ alkyl, substituted C₁-C₃ alkyl, C₁-C₃ alkoxy, CH₂—X—CH₃, or
(CH₂)ₘR³;
R² is H, COOH, COOR⁴, (CH₂)ₙOH, CH(OH)R⁵, CH₂OR⁵, C(O)NH₂, C(O)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶), C(O)NR⁵NH(C(O)R⁶), C(O)NR⁵NR⁶(C(O)R⁷), CN, or C(O)R⁵;
R³ is C₁-C₃ alkyl, benzyl, substituted C₁-C₃ alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, O(CH₂)ₚO(CH₂)_{q}O(CH₂)ᵣCH₃ or NR⁸C(O)R⁹;
R4 is C₁-C₆ alkyl or (CH₂CH₂O)ₙCH₃;
R⁵, R⁶, R⁷, R⁸, R⁹, R¹⁰, and R¹¹ are independently alkyl or substituted alkyl;
R¹² is H, alkyl, or substituted alkyl;
R¹³ is H, OR¹⁰, alkyl, or substituted alkyl;
X is O or NH;
Y is O or S;
m is an integer selected from 0-8;
each of n, p and q is 1, 2 or 3; and
r is 0, 1 or 2.

In another embodiment, the ibogaine derivative is represented by Formula Ii:

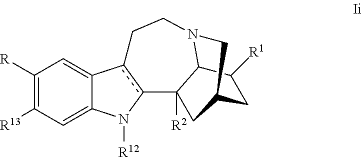

or a pharmaceutically acceptable salt and/or solvate thereof,
wherein
R is H, halo, C₁-C₃ alkyl, substituted C₁-C₃ alkyl, OR¹⁰, NH₂, NHR¹⁰, NR¹⁰R¹¹, NHC(O)R¹⁰, or NR¹⁰C(O)R¹¹;
R¹ is H, C₁-C₃ alkyl, substituted C₁-C₃ alkyl, C₁-C₃ alkoxy, CH₂—X—CH₃, or (CH₂)ₘR³;
R² is H, COOH, COOR⁴, (CH₂)ₙOH, CH(OH)R⁵, CH₂OR⁵, C(O)NH₂, C(O)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶), C(O)NR⁵NH(C(O)R⁶), C(O)NR⁵NR⁶(C(O)R⁷), CN, or C(O)R⁵;
R³ is C₁-C₃ alkyl, benzyl, substituted C₁-C₃ alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, O(CH₂)ₚO(CH₂)_{q}O(CH₂)ᵣCH₃ or NR⁸C(O)R⁹;
R⁴ is C₁-C₆ alkyl or (CH₂CH₂O)ₙCH₃;
R⁵, R⁶, R⁷, R⁸, R⁹, R¹⁰, and R¹¹ are independently alkyl or substituted alkyl;
R¹² is H, alkyl, or substituted alkyl;
R¹³ is H, OR¹⁰, alkyl, or substituted alkyl;
X is O or NH;
Y is O or S;
m is an integer selected from 0-8;
each of n, p and q is 1, 2 or 3; and
r is 0, 1 or 2.

In one embodiment, the compound is of Formula IA:

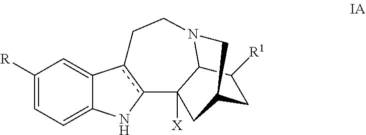

wherein
R is hydrogen or C₁-C₃ -alkoxy,
R¹ is hydrogen, C₁-C₃ -alkyl, C₁-C₃ alkoxy, or CH₂—Y—CH₃ where Y is O or NH, and
X is H, COOH, or COOR², where R² is C₁-C₆ alkyl or (CH₂CH₂O)ₙCH₃, where n=1 to 3.

In one embodiment a compound utilized herein is represented by, or ibogaine as used herein is replaced by, a compound Formula II:

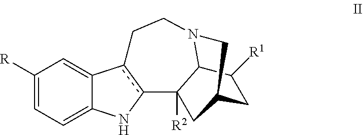

or a pharmaceutically acceptable salt and/or solvate thereof,
wherein
R is hydrogen or C₁-C₃ alkoxy;
R¹ is hydrogen, C₁-C₃ alkyl, C₁-C₃ alkoxy, (CH₂)ₘOC(O)alkyl, (CH₂)ₘOH, (CH₂)ₘOalkyl, (CH₂)ₘO(CH₂)ₚO(CH₂)_{q}O(CH₂)ᵣCH₃ or CH₂—Y—CH₃ where each of m, p and q is 1, 2 or 3; and r is 0, 1 or 2, Y is O or NH; and
R² is H, (CH₂)ₙOH, COOH, or COOR⁴, where R⁴ is C₁-C₆ alkyl or (CH₂CH₂O)ₙCH₃, where n is 1, 2, or 3.

In one embodiment, the ibogaine derivative is represented by Formula II:

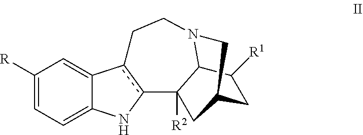

or a pharmaceutically acceptable salt and/or solvate thereof,
wherein
R is OCH₃;
R¹ is CH₂CH₃; and
R² is COOR⁴, where R⁴ is (CH₂CH₂O)ₙCH₃, where n is 1.

In another embodiment, ibogaine or a pharmaceutically acceptable salt and/or solvate thereof is utilized. In another embodiment, ibogaine or a pharmaceutically acceptable salt and/or solvate thereof is utilized. In another embodiment, the ibogaine, ibogaine derivative, is chosen from the group consisting of ibogaine, coronaridine, ibogamine, voacangine, 18-methoxycoronaridine, 2-methoxyethyl-18-methoxycoronaridinate, 18-methylaminocoronaridine or a pharmaceutically acceptable salt and/or solvate thereof.

In another embodiment, the compound utilized herein is chosen from the group consisting of ibogaine, coronaridine, ibogamine, voacangine, 18-methoxycoronaridine, 2-methoxyethyl-18-methoxycoronaridinate, 18-methylaminocoronaridine and a pharmaceutically acceptable salt and/or solvate.

In another embodiment, the compound utilized herein is selected from the group consisting of 16-hydroxymethyl-18-hydroxyibogaline. 16-hydroxymethyl-18-methoxyibogaline, 16-ethoxycarbonyl-18-hydroxyibogaline laurate, and 16-ethoxycarbonyl-18-hydroxyibogaline methoxyethoxymethyl ether and a pharmaceutically acceptable salt and/or solvate thereof.

When replacing ibogaine, the compounds of formula I, II, and subformulas thereof as utilized herein exclude ibogaine.

In a preferred embodiment, the compound utilized herein is:

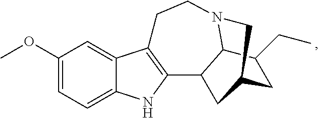

a pharmaceutically acceptable salt thereof, or a solvate of each thereof.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

### I. Definitions

Unless defined otherwise, all technical and scientific terms used herein have the same meaning as commonly understood by one of ordinary skill in the art to which this invention belongs. As used herein the following terms have the following meanings.

The term “about” when used before a numerical designation, e.g., temperature. time, amount, concentration, and such other, including a range, indicates approximations which may vary by (+) or (−) 10%, 5% or 1%, or any subrange or subvalue there between.

“Administration” refers to introducing ibogaine, ibogaine derivative, or pharmaceutically acceptable salt or solvate thereof into a patient. Typically, an effective amount is administered, which amount can be determined by the treating physician or the like. Any route of administration, such as oral, topical, subcutaneous. peritoneal, intra-arterial, inhalation, vaginal, rectal, nasal, introduction into the cerebrospinal fluid, or instillation into body compartments can be used. The ibogaine, ibogaine derivative, or pharmaceutically acceptable salt or solvate thereof may be administered by direct blood stream delivery, e.g. sublingual, intranasal, or intrapulmonary administration.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of ibogaine, ibogaine derivative, or pharmaceutically acceptable salt or solvate thereof one, two, three, or more times per day. Administration may be via transdermal patch, gum, lozenge, sublingual tablet, intranasal, intrapulmonary, oral administration, or other administration.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, [symbol] is a single bond or a double bond.

“Ibogaine” as a specific compound refers to the compound:

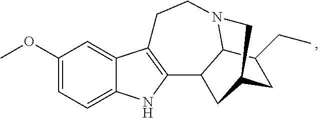

as well as ibogaine derivatives or pharmaceutically acceptable salts and pharmaceutically acceptable solvates thereof. It should be understood that where “ibogaine” is mentioned herein, one more polymorphs of ibogaine can be utilized and are contemplated. Ibogaine is isolated from Tabernanth iboga, a shrub of West Africa. Ibogaine can also be synthesized using known methods. See, e.g., Büchi, et al. (1966), J. Am. Chem Society. 88(13), 3099-3109. Unless specified otherwise, “ibogaine” as used herein refers to ibogaine, ibogaine derivative, or a pharmaceutically acceptable salt and/or solvate thereof.

In some embodiments, the ibogaine or ibogaine derivative is represented by Formula I:

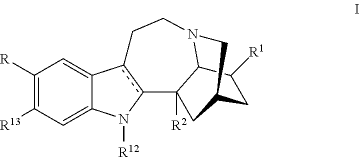

or a pharmaceutically acceptable salt and/or solvate thereof,
wherein
R is H, halo, C₁-C₃ alkyl, substituted C₁-C₃ alkyl, OR¹⁰, NH₂, NHR¹⁰, NR¹⁰R¹¹, NHC(O)R¹⁰, or NR¹⁰C(O)R¹¹;
R¹ is H, C₁-C₃ alkyl, substituted C₁-C₃ alkyl, C₁-C₃ alkoxy, CH₂—X—CH₃, or (CH₂)ₘR³;
R² is H, COOH, COOR⁴, (CH₂)ₙOH, CH(OH)R⁵, CH₂OR⁵, C(O)NH₂, C(O)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶), C(O)NR⁵NH(C(O)R⁶), C(O)NR⁵NR⁶(C(O)R⁷), CN, or C(O)R⁵;
R³ is C₁-C₃ alkyl, benzyl, substituted C₁-C₃ alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, O(CH₂)ₚO(CH₂)_{q}O(CH₂)ᵣCH₃ or NR⁸C(O)R⁹;
R⁴ is C₁-C₆ alkyl or (CH₂CH₂O)ₘCH₃;
R⁵, R⁶, R⁷, R⁸, R⁹, R¹⁰, and R¹¹ are independently alkyl or substituted alkyl;
R¹² is H, alkyl, or substituted alkyl;
R¹³ is H, OR¹⁰, alkyl, or substituted alkyl;
X is O or NH;
Y is O or S;
m is an integer selected from 0-8;
each of n, p and q is 1, 2 or 3; and
r is 0, 1 or 2.

In some embodiments, the ibogaine or ibogaine derivative is represented by Formula II:

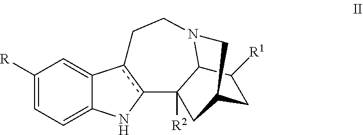

or a pharmaceutically acceptable salt and/or solvate thereof,
wherein
R is hydrogen or C₁-C₃ alkoxy,
R¹ is hydrogen, C₁-C₃ alkyl, C₁-C₃ alkoxy, (CH₂)ₘOC(O)alkyl, (CH₂)ₘOH, (CH₂)ₘOalkyl, (CH₂)ₘO(CH₂)ₚO(CH₂)_{q}O(CH₂)ᵣCH₃ or CH₂—Y—CH₃ where each of m, p and q is 1, 2 or 3; and r is 0, 1 or 2, Y is O or NH, and
R² is H, (CH₂)ₙOH, COOH, or COOR⁴, where R⁴ is C₁-C₆ alkyl or (CH₂CH₂O)ₙCH₃, where n is 1, 2, or 3.

In one embodiment, R is methoxy. In one embodiment, R¹ is ethyl. In one embodiment, R¹ is methoxy. In one embodiment, R¹ is CH₂—Y—CH; where Y is O. In one embodiment, R¹ is CH₂—Y—CH₃ where Y is NH. In one embodiment, R² is hydrogen. In one embodiment, In one embodiment, R² is COOR⁴ and R⁴ is methyl. In one embodiment, n=1. In a preferred embodiment, R, R¹ and R² are all not hydrogen. In one embodiment, when R is methoxy and R¹ is hydrogen, then R² is COOH or COOR⁴. In another embodiment, when R is methoxy and R¹ is hydrogen, then X is COOR⁴ where R⁴ is (CH₂CH₂O)CH₃.

In one embodiment, R¹² is hydrogen.

In one embodiment, R¹ is H. In one embodiment, R¹ is C₁-C₃ alkyl, such as ethyl. In one embodiment, R¹ is CH₂CH₂OH. In one embodiment. R¹ is CH₂CH₂OCH₃. In one embodiment, R¹ is CH₂CH₂OCH₂Ph. In one embodiment, R¹ is CH₂CH₂OC(O)alkyl. In one embodiment, R¹ is CH₂CH₂O(CH₂)ₚO(CH₂)_{q}O(CH₂)ᵣCH₃.

In one embodiment, R² is CH₂OH and CH(OH)R⁵. In one embodiment, R² is CH₂OR⁵. In one embodiment, R² is CO₂R⁵. In one embodiment, R² is C(O)NH₂, C(O)NHR⁵, or C(O)NR⁵R⁶. In one embodiment, R² is C(O)NHNH₂, C(O)NHNHR⁵. C(O)NR⁵NH₂, C(O)NHNR⁵R⁶, C(O)NH⁵NHR⁶, or C(O)NR⁵NR⁶R⁷. In one embodiment, R² is C(O)NHNH(C(O)R⁵), C(O)NHNR⁵(C(O)R⁶), C(O)NR⁵NH(C(O)R⁶), or C(O)NR⁵NR⁶(C(O)R⁷). In one embodiment, R² is C(O)R⁵.

In one embodiment, the compound is of Formula IA:

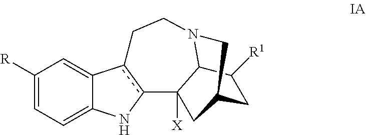

wherein
R is hydrogen or C₁-C₃ -alkoxy,
R¹ is hydrogen, C₁-C₃ -alkyl, C₁-C₃ alkoxy, or CH₂—Y—CH₃ where Y is O or NH, and
X is H, COOH, or COOR², where R² is C₁-C₆ alkyl or (CH₂CH₂O)ₙCH₃, where n=1 to 3.

In another embodiment, the ibogaine derivative is represented by Formula II:

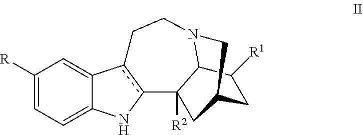

or a pharmaceutically acceptable salt and/or solvate thereof,
wherein
R is OCH₃;
R¹ is CH₂CH₃; and
R² is COOR⁴, where R⁴ is (CH₂CH₂O)ₙCH₃, where n is 1.

When replacing ibogaine, the compounds of formula I, II, and subformulas thereof as utilized herein exclude ibogaine.

In a preferred embodiment, the compound utilized herein is:

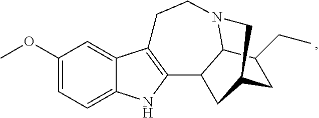

a pharmaceutically acceptable salt thereof, or a solvate of each thereof.

In some embodiments, the ibogaine or ibogaine derivative is selected from:

| Name | Structure |
|---|---|
| coronaridine | 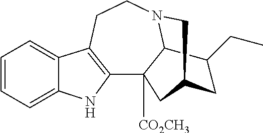 |
| 18-hydroxycoronaridine | 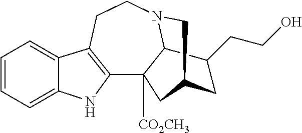 |
| 18-methoxycoronaridine | 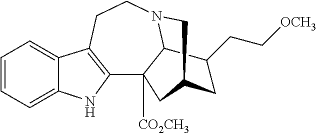 |
| 18-benzyloxycoronaridine | 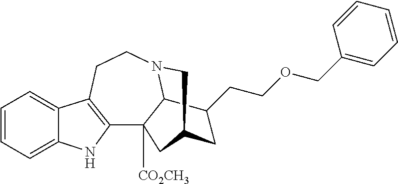 |
| 18-hydroxycoronaridine laurate | 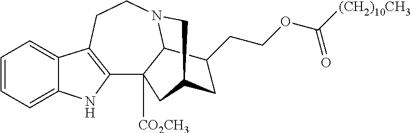 |
| 18-hydroxycoronaridine methoxyethoxymethyl ether | 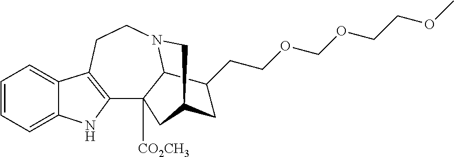 |
| 18-hydroxycoronaridine acetate | 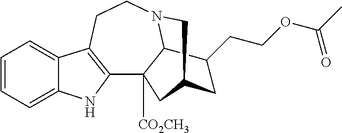 |
| voacangine | 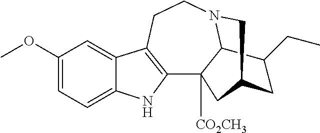 |
| 18-hydroxyvoacangine | 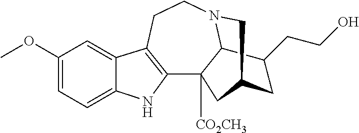 |
| 18-methoxyvoacangine | 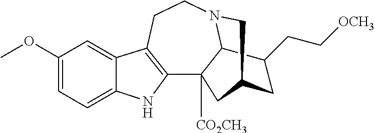 |
| 18-benzyloxyvoacangine | 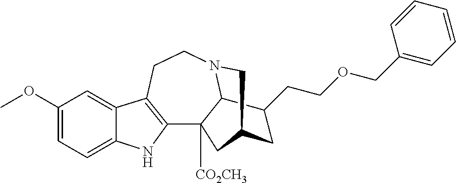 |
| 18-hydroxyvoacangine laurate | 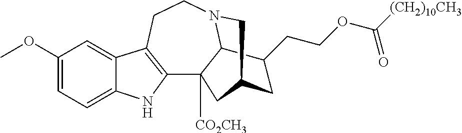 |
| 18-hydroxyvoacangine acetate | 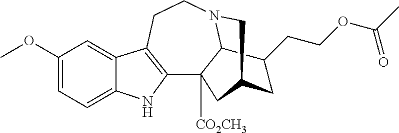 |
| 18-hydroxyvoacangine methoxyethoxymethyl ether | 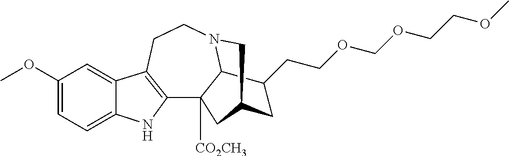 |
| conopharyngine | 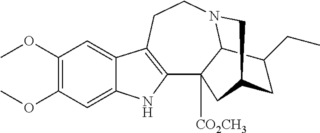 |
| 18-hydroxyconopharyngine | 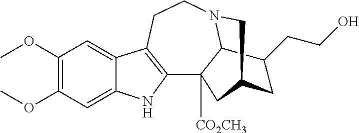 |
| 18-rnethoxyconopharyngine | 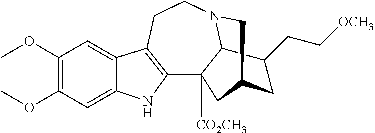 |
| 18-benzyloxyconopharyngine | 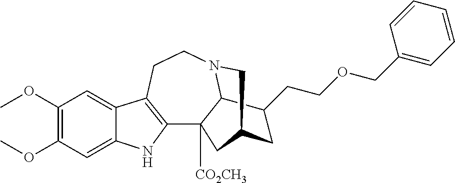 |
| 18-hydroxyconopharyngine laurate | 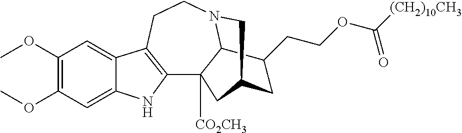 |
| 18-hydroxyconopharyngine acetate | 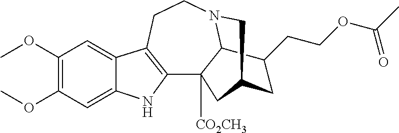 |
| 18-hydroxyconopharyngine methoxyethoxymethyl ether | 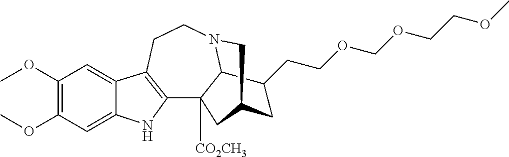 |
| ibogamine | 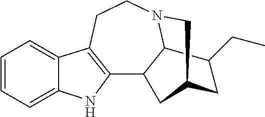 |
| 16-ethoxycarbonyl-18- hydroxyibogamine | 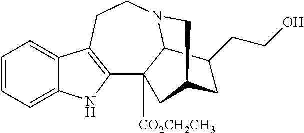 |
| 16-hydroxymethyl-18- hydroxyibogamine | 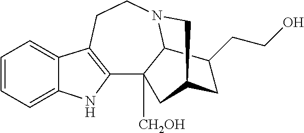 |
| 16-ethoxycarbonyl-18- methoxyibogamine | 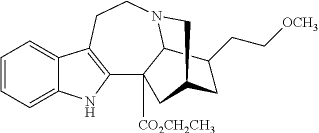 |
| 16-hydroxymethyl-18- methoxyibogamine | 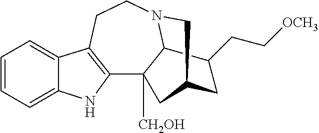 |
| 16-ethoxycarbonyl-18- benzyloxyibogamine | 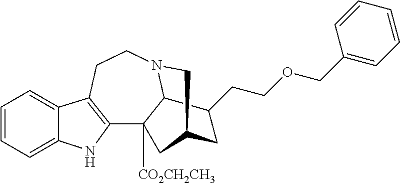 |
| 16-ethoxycarbonyl-18- hydroxyibogamine laurate | 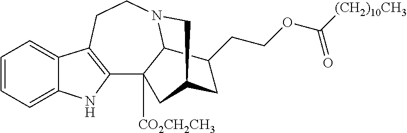 |
| 16-ethoxycarbonyl-18- hydroxyibogamine acetate | 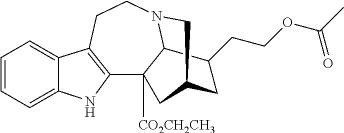 |
| 16-ethoxycarbonyl-18- hydroxyibogamine methoxyethoxymethyl ether | 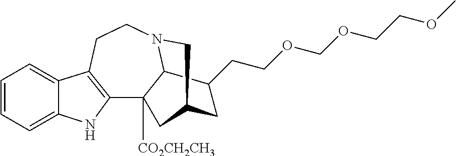 |
| ibogaine | 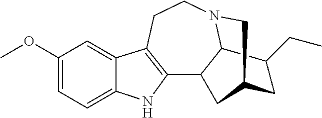 |
| 16-ethoxycarbonyl-18- hydroxyibogaine | 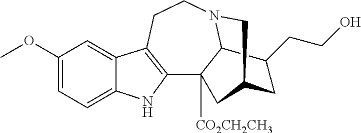 |
| 16-hydroxymethyl-18- hydroxyibogaine | 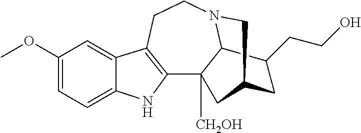 |
| 16-ethoxycarbonyl-18- methoxyibogaine | 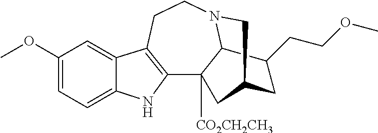 |
| 16-hydroxymethyl-18- methoxyibogaine | 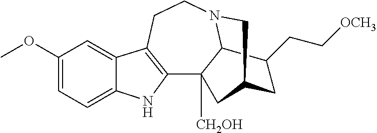 |
| 16-ethoxycarbonyl-18- benzyloxyibogaine | 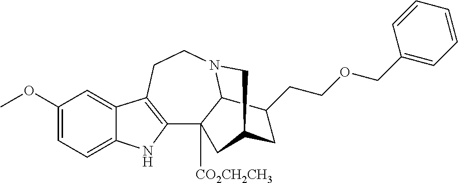 |
| 16-ethoxycarbonyl-18- hydroxyibogaine laurate | 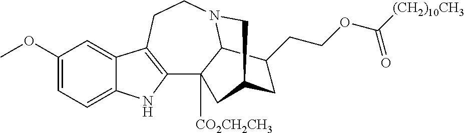 |
| 16-ethoxycarbonyl-18- hydroxyibogaine acetate | 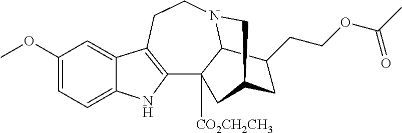 |
| 16-ethoxycarbonyl-18- hydroxyibogaine methoxyethoxymethyl ether | 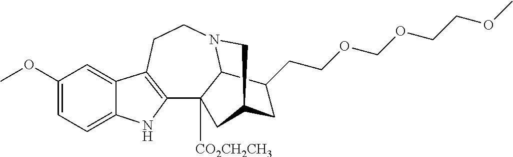 |
| ibogaline | 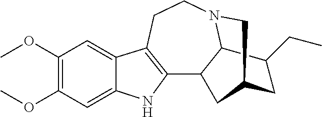 |
| 16-ethoxycarbonyl-18- hydroxyibogaline | 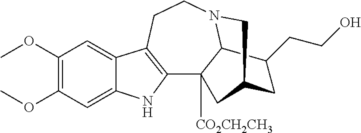 |
| 16-hydroxymethyl-18- hydroxyibogaline | 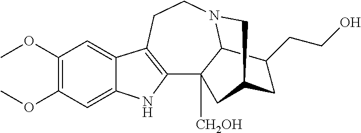 |
| 16-ethoxycarbonyl-18- methoxyibogaline | 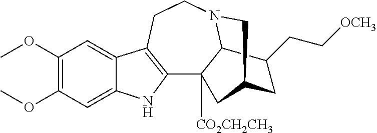 |
| 16-hydroxymethyl-18- methoxyibogaline | 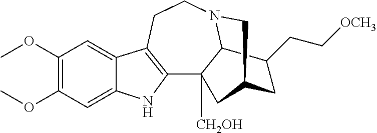 |
| 16-ethoxycarbonyl-18- benzyloxyibogaline | 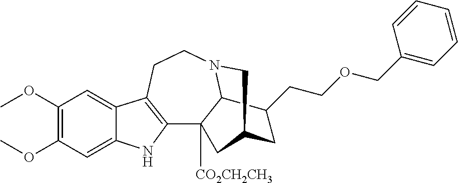 |
| 16-ethoxycarbonyl-18- hydroxyibogaline laurate | 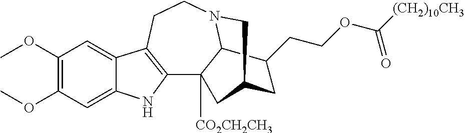 |
| 16-ethoxycarbonyl-18- hydroxyibogaline acetate | 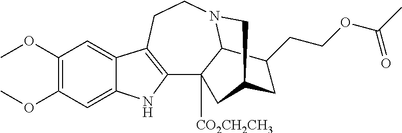 |
| 16-ethoxycarbonyl-18- hydroxyibogaline methoxyethoxymethyl ether | 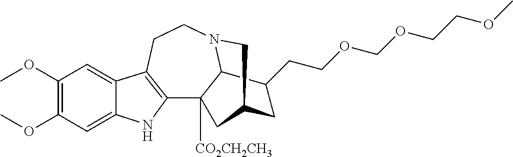 |

and pharmaceutically acceptable salts and/or solvates thereof.

This invention is not limited to any particular chemical form of the compounds, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like.

The term “ibogaine derivatives” refers to a compound of formula I, solvates thereof, or pharmaceutically acceptable salts of each thereof. In one embodiment, the ibogaine derivative is represented by Formula I:

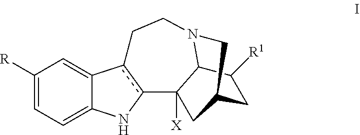

or a pharmaceutically acceptable salt thereof,
wherein
R is hydrogen or C₁-C₃ -alkoxy,
R¹ is hydrogen, C₁-C₃-alkyl, C₁-C₃ alkoxy, or CH₂—Y—CH₃ where Y is O or NH, and
X is H, COOH, or COOR², where R² is C₁-C₆ alkyl or (CH₂CH₂O)ₙCH₃, where n=1 to 3.

In one embodiment, R is methoxy. In one embodiment, R¹ is methoxy. In one embodiment, R¹ is CH₂—Y—CH where Y is O. In one embodiment, R¹ is CH₂—Y—CH₃ where Y is NH. In one embodiment, R² is methyl. In one embodiment, n=1. In a preferred embodiment, R, R¹ and X are all not hydrogen. In one embodiment, when R is methoxy and R¹ is hydrogen, then X is COOH or COOR². In another embodiment, when R is methoxy and R¹ is hydrogen, then X is COOR² where R² is (CH₂CH₂O)CH₃.

As used herein, the term “alkyl” refers to monovalent saturated aliphatic hydrocarbyl groups having from 1 to 12 carbon atoms, 1 to 10 carbon atoms, preferably 1 to 6 carbon atoms, and more preferably 1 to 3 carbon atoms. This term includes, by way of example, linear and branched hydrocarbyl groups such as methyl (CH₂—), ethyl (CH₃CH₂—), n-propyl (CH₃CH₂CH₂—), isopropyl ((CH₃)₂CH—), n-butyl (CH₃CH₂CH₂CH₂—, isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “Cₓ alkyl” refers to an alkyl group having x carbon atoms, wherein x is an integer, for example, C₃ refers to an alkyl group having 3 carbon atoms.

“Substituted alkyl” refers to an alkyl group having from 1 to 5, preferably 1 to 3, or more preferably 1 to 2 substituents selected from the group consisting of alkoxy, R²⁰—C(O)—, —NR²⁰C(O)R²⁰, R²⁰—C(O)O—, —NR²⁰R²⁰, —C(O)NR²⁰R²⁰, —C(S)NR²⁰R²⁰, —NR²⁰C(O)NR²⁰R²⁰, —-NR²⁰C(S)NR²⁰R²⁰, —O—C(O)NR²⁰R²⁰, —S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl, SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle; and each R²¹ is independently selected from the group consisting of alkyl, cycloalkyl, aryl, heteroaryl, and heterocycle.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkoxy, —C(O)—R²⁰, —NR²⁰C(O)R²⁰, R²⁰—C(O)O—, —NR²⁰R²⁰, —C(O)NR²⁰R²⁰, —C(S)NR²⁰R²⁰, —NR²⁰C(O)NR²⁰R²⁰, —NR²⁰C(S)NR²⁰R²⁰, —O—C(O)NR²⁰R²⁰, —S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl, SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle: and each R²¹ is independently selected from the group consisting of alkyl, cycloalkyl, aryl, heteroaryl, and heterocycle.

“Cyano” refers to the group —CN.

“Cycloalkyl” refers to cyclic alkyl groups of from 3 to 10 or 3 to 8 carbon atoms having single or multiple cyclic rings including fused, bridged. and spiro ring systems. One or more of the rings can be aryl. heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbomnyl, and spirobicyclo groups such as spiro[4.5]dec-8-yl.

“Substituted cycloalkyl” refers to a cycloalkyl group having from 1 to 5 or preferably 1 to 3 substituents selected from the group consisting of oxo, thione, alkyl, substituted alkyl, alkoxy, —C(O)—R²⁰, —NR²⁰C(O)R²⁰, R²⁰—C(O)O—, —NR²⁰R²⁰, —C(O)NR²⁰R²⁰, —C(S)NR²⁰R²⁰, —NR²⁰C(O)NR²⁰R²⁰, —NR²⁰C(S)NR²⁰R²⁰, —O—C(O)NR²⁰R²⁰, —S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl, SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle; and each R²¹ is independently selected from the group consisting of alkyl, cycloalkyl, aryl, heteroaryl, and heterocycle.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Heteroaryl” refers to an aromatic group of from 5 to 14 ring atoms, including from 1 to 10 carbon atoms and 1 to 4 heteroatoms selected from the group consisting of oxygen, nitrogen and sulfur. In some embodiments, heteroaryl comprises 5, 6, or 7 ring atoms, including 1 to 4 heteroatoms. Such heteroaryl groups can have a single ring (e.g., pyridyl, pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heterocycle” or “heterocyclic” or “heterocycloalkyl” or “heterocyclyl” refers to a saturated or partially saturated, but not aromatic, group having from 3 to 14 ring atoms, including from 1 to 10 ring carbon atoms and from 1 to 4 ring heteroatoms selected from the group consisting of nitrogen, sulfur, or oxygen. In some embodiments, heteroaryl comprises 3, 4, 5, 6 or 7 ring atoms, including 1 to 4 heteroatoms. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

In one embodiment, the ibogaine derivative is:

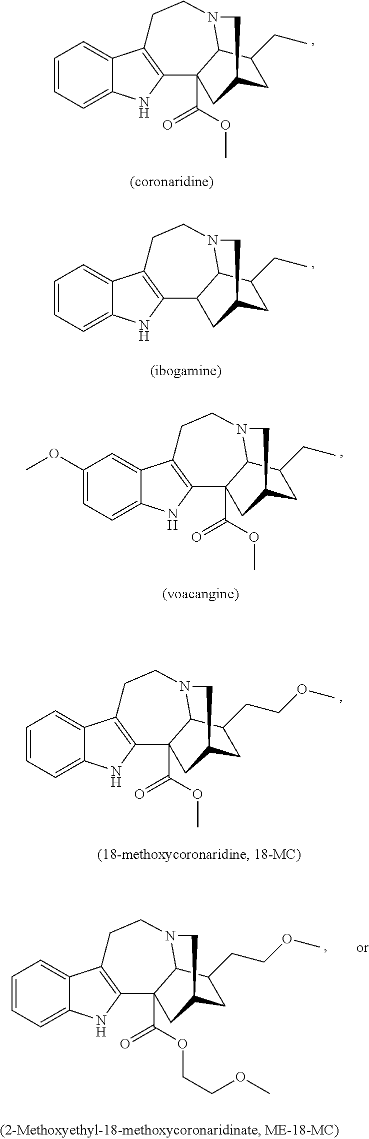

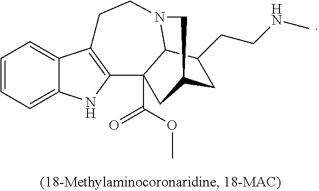

“Pharmaceutically acceptable composition” refers to a composition that is suitable for administration to a mammal, particularly, a human. Such compositions include various excipients, diluents, carriers, and such other inactive agents well known to the skilled artisan.

“Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

A “pharmaceutically acceptable solvate” or “hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

As used herein the term “solvate” is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C₁-C₆ alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present invention. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present invention may absorb moisture, may include one or more molecules of water in the formed crystal, and thus become a hydrate. Even when such hydrates are formed, they are included in the term “solvate”. Solvate also is meant to include such compositions where another compound or complex co-crystallizes with the compound of interest.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt. solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of ibogaine, ibogaine derivative, or pharmaceutically acceptable salt or solvate thereof in the context of treating opioid or opioid-like drug dependency, refers to an amount of compound that attenuates the dependency and/or symptoms of acute withdrawal for at least 2 hours beyond control (placebo), at least 5 hours beyond control, and preferably at least 10 hours beyond control.

A “therapeutic level” of a drug is an amount of ibogaine, ibogaine derivative, or pharmaceutically acceptable salt or solvate thereof that is sufficient to treat opioid or opioid-like drug addiction or to treat, prevent, or attenuate acute withdrawal symptoms, but not high enough to pose any significant risk to the patient. Therapeutic levels of drugs can be determined by tests that measure the actual concentration of the compound in the blood of the patient. This concentration is referred to as the “serum concentration.”

As defined herein, a “prophylactically effective amount” of a drug is an amount, typically less than the therapeutically effective amount, that provides attenuation and/or prevention of a disease or disorder or symptoms of a disease or disorder in a patient. For example, the prophylactically effective amount of the compound is expected to be less than the therapeutically effective amount because the level of inhibition does not need to be as high in a patient who is no longer physically addicted to nicotine. For example, a prophylactically effective amount is preferably 90%, 80%, 70%, 60%, 50%, 40%, 30%, 20%, or 10% less than a therapeutically effective amount. However, a prophylactically effective amount may be the same as the therapeutically effective amount, for example when a patient who is physically addicted to nicotine is administered ibogaine, ibogaine derivative, or pharmaceutically acceptable salt or solvate thereof to attenuate cravings for a period of time when nicotine use is not feasible. The prophylactically effective amount may vary for different a diseases or disorders or symptoms of different diseases or disorders.

As defined herein, a “maintenance amount” of a drug is an amount, typically less than the therapeutically effective amount that provides attenuation and/or prevention of post-acute withdrawal syndrome in a patient. The maintenance amount of the compound is expected to be less than the therapeutically effective amount because the level of inhibition does not need to be as high in a patient who is no longer physically addicted to opioid or opioid-like drug. For example, a maintenance amount is preferably 80%, 70%, 60%, 50%, 40%, 30%, 20%, or 10% less than a therapeutically effective amount, or any subvalue or subrange there between.

“Treatment,” “treating,” and “treat” are defined as acting upon a disease. disorder, or condition with an agent to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to: treating opioid or opioid-like drug addiction; treating, preventing, and/or attenuating acute withdrawal symptoms; treating, preventing, and/or attenuating long-term (post-acute) withdrawal symptoms; and preventing relapse of opioid or opioid-like drug use

As used herein, the term “patient” refers to a mammal and include humans and non-human mammals.

As used herein, the term “opiate” refers to naturally-occurring alkaloids found in the opium poppy. These include codeine, morphine, oripavine, pseudomorphine, and thebaine. Also included are opium, opium poppy, poppy straw, and extracts and concentrates thereof.

As used herein, the term “opioid” refers to naturally-occurring opiates and synthetic or semi-synthetic opioids that have psychoactive effects. Non-limiting examples include acetyl-alpha-methylphentanyl, acetylmethadol, alfentanil, allylprodine, alphacetylmethadol, alphamethadol, alpha-methylfentanyl, alpha-methylthiofentanyl, alphaprodine, anileridine, benzylmorphine, benzethidine, betacetylmethadol, beta-hydroxyfentanyl, beta-hydroxy-3-methylfentanyl, betameprodine, betacetylmethadol, beta-hydroxy fentanyl, beta-hydroxy-3-methylfentanyl, betameprodine, betamethadol, betaprodine, bezitramide, buprenorphine, butorphanol, carfentanil, clonitazene, codeine, desomorphine, dextromoramide, dextropropoxyphene, dezocine, diampromide, diamorphone, diethylthiambutene, dihydrocodeine, dihydroetorphine, dihydromorphine, dimenoxadol, dimepheptanol, dimethyl-thiambutene, dioxaphetyl butyrate, diphenoxylate, difenoxin, dipipanone, eptazocine, ethoheptazine, ethylmethylthiambutene, ethylmorphine, etonitazene, etorphine, etoxeridine, fentanyl, furethidine, heroin, hydrocodone, hydromorphone, hydroxypethidine, isomethadone, ketobemidone, levo-alphacetylmethadol, levomethorphan, levorphanol, levophenacylmorphan, levomoramide, lofentanil, loperamide, laudanum, meperidine, meptazinol, metazocine, methadone, 3-methylfentanyl, 3-methylthiofentanyl, metopon, morphine, morpheridine, MPPP (1-methyl-4-phenyl-4-propionoxypiperidine), myrophine, narceine, nicomorphine, noracymethadol, norlevorphanol, normethadone, nalorphine, nalbuphene, normorphine, norpipanone, opium, oxycodone, oxymorphone, papaveretum, para-fluorofentanyl, paregoric, PEPAP (1-(-2-phenethyl)-4-phenyl-4-acetoxypiperidine), pentazocine, phenadoxone, phenampromide, phenomorphan, phenazocine, phenoperidine, piminodine, piritramide, propheptazine, promedol, properidine, propiram, propoxyphene, racemoramide, racemethorphan, racemorphan, remifentanil, sufentanil, tapentadol, thebaine, thiofentanyl, tilidine, tramadol, trimeperidine, mixtures of any of the foregoing, salts of any of the foregoing, derivatives of any of the foregoing, and the like. The term opioids also encompasses opioid intermediates, including 4-cyano-2-dimethylamino-4,4-diphenyl butane. 2-methyl-3-morpholino-1,1-diphenylpropane-carboxylic acid, 4-cyano-1-methyl-4-phenylpiperidine, ethyl-4-phenylpiperidine-4-carboxylate, and 1-methyl-4-phenylpiperidine-4-carboxylic acid. Many opioids are Schedule I or Schedule II drugs in the US.

As used herein, the term “opioid-like drug” refers to any illicit drug that binds to one or more opioid receptor and causes opioid-like addiction. Acute and long-term withdrawal symptoms from cessation of use of such drugs may be similar to those from cessation of opioids. Opioid-like drugs include amphetamine, methamphetamine, ketamine, and cocaine.

As used herein, the term “QT interval” refers to the measure of the time between the start of the Q wave and the end of the T wave in the electrical cycle of the heart. Prolongation of the QT interval refers to an increase in the QT interval.

As used herein, the terms “addiction” and “dependence” are used interchangeably to refer to the patient's inability to stop using the opioid or opioid-like drug, even when it would be in his/her best interest to stop. The DSMIV-TR criteria for dependency include:
Dependence or significant impairment or distress, as manifested by 3 or more of the following during a 12 month period:
1. Tolerance or markedly increased amounts of the substance to achieve intoxication or desired effect or markedly diminished effect with continued use of the same amount of substance
2. Withdrawal symptoms or the use of certain substances to avoid withdrawal symptoms
3. Use of a substance in larger amounts or over a longer period than was intended
4. Persistent desire or unsuccessful efforts to cut down or control substance use
5. Involvement in chronic behavior to obtain the substance, use the substance, or recover from its effects
6. Reduction or abandonment of social, occupational or recreational activities because of substance use
7. Use of substances even though there is a persistent or recurrent physical or psychological problem that is likely to have been caused or exacerbated by the substance.

The term “solvate” as used herein refers to complexes with solvents in which the compound is reacted or from which the compound is precipitated or crystallized. For example, a complex with water is known as a “hydrate.” Solvates of are within the scope of the invention. It will be appreciated by those skilled in organic chemistry that many organic compounds can exist in more than one crystalline form. For example, crystalline form may vary based on the solvate used. Thus, all crystalline forms of the compounds or the pharmaceutically acceptable solvates thereof are within the scope of the present invention.

The term “dose” refers to a range of ibogaine, ibogaine derivative, or pharmaceutical salt or solvate thereof that provides a therapeutic serum level of ibogaine when given to a patient in need thereof. The dose is recited in a range, for example from about 20 mg to about 120 mg, and can be expressed either as milligrams or as mg/kg body weight. The attending clinician will select an appropriate dose from the range based on the patient's weight, age, degree of addiction, health, and other relevant factors, all of which are well within the skill of the art.

The term “unit dose” refers to a dose of drug that is given to the patient to provide therapeutic results. independent of the weight of the patient. In such an instance, the unit dose is sold in a standard form (e.g., 20 mg tablet). The unit dose may be administered as a single dose or a series of subdoses. In some embodiments, the unit dose provides a standardized level of drug to the patient, independent of weight of patient. Many medications are sold based on a dose that is therapeutic to all patients based on a therapeutic window. In such cases, it is not necessary to titrate the dosage amount based on the weight of the patient.

### II. Compositions

As will be apparent to the skilled artisan upon reading this disclosure, this invention provides compositions for treating nicotine addiction in a subject, comprising ibogaine, ibogaine derivatives, or pharmaceutically acceptable salts and/or solvates of each thereof. In another aspect, this invention further provides compositions for treating, attenuating, or preventing nicotine cravings in a subject, comprising ibogaine, ibogaine derivative, or pharmaceutically acceptable salt or solvate thereof.

This invention is not limited to any particular chemical form of the compounds, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like. Dosing schemes are discussed in further detail below in the subsection titled “Dosing and Routes of Administration.”

In one aspect, the invention provides a pharmaceutical composition comprising a therapeutically or prophylactically effective amount of ibogaine, ibogaine derivative, or pharmaceutically acceptable salt or solvate thereof and a pharmaceutically acceptable excipient, wherein the therapeutically or prophylactically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to less than 10 μg per kg body weight per day. In some aspects, the therapeutically or prophylactically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 10 μg per kg body weight per day. In some aspects, the therapeutically or prophylactically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 10 μg per kg body weight per day. In some aspects, the composition is formulated for administration once per day. In some aspects, the composition is formulated for administration two or more times per day.

In some embodiments, the composition is formulated for sublingual, intranasal, or intrapulmonary delivery. These routes of administration are discussed in further detail below in the subsection titled “Dosing and Routes of Administration.”

### III. Methods of the Invention

As will be apparent to the skilled artisan upon reading this disclosure, the present invention provides a method for treating opioid or opioid-like drug, abuse including acute and post-acute withdrawal symptoms, in a patient addicted to opioid or opioid-like drug, comprising administering to the patient a dosage of ibogaine, ibogaine derivative, or pharmaceutically acceptable salt or solvate thereof.

### Therapeutic Administration

In one aspect, this invention relates to treatment of acute withdrawal from an opioid or opioid-like drug in an addicted patient comprising administration of a therapeutically effective amount of ibogaine, ibogaine derivative, or pharmaceutically acceptable salt or solvate thereof.

In one aspect, this invention relates to a method for treating opioid or opioid-like drug abuse in an addicted patient, comprising administering to the patient a dosage of ibogaine, ibogaine derivative, or pharmaceutically acceptable salt or solvate thereof that provides an average serum concentration of about 50 ng/mL to about 850 ng/mL, said concentration being sufficient to inhibit or ameliorate said abuse while maintaining a QT interval of less than about 500 ms during said treatment.

In one aspect, this invention relates to a method for attenuating withdrawal symptoms in a human patient susceptible to such symptoms due to opioid or opioid-like drug addiction, comprising administering to the patient a dosage of ibogaine, ibogaine derivative, or pharmaceutically acceptable salt or solvate thereof that provides an average serum concentration of about 50 ng/mL to about 400 ng/mL, said concentration being sufficient to attenuate said symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment. In one embodiment, the withdrawal symptoms are symptoms of acute withdrawal.

In one aspect, this invention relates to a method for attenuating withdrawal symptoms in a human patient susceptible to such symptoms due to opioid or opioid-like drug addiction, comprising administering to the patient a dosage of ibogaine, ibogaine derivative, or pharmaceutically acceptable salt or solvate thereof that provides an average serum concentration of about 50 ng/mL to about 400 ng/mL, said concentration being sufficient to attenuate said symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment. In one embodiment, the withdrawal symptoms are symptoms of acute withdrawal.

In one aspect, the invention provides administering a pharmaceutical composition comprising a pharmaceutically effective amount of ibogaine, ibogaine derivative, or pharmaceutically acceptable salt or solvate thereof and a pharmaceutically acceptable excipient, wherein the therapeutically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 100 μg per kg body weight per day. In some aspects, the therapeutically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 50 μg per kg body weight per day. In some aspects, the therapeutically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 10 μg per kg body weight per day. In some aspects, the therapeutically effective amount of ibogaine, ibogaine derivative, or pharmaceutically acceptable salt or solvate thereof is an amount that delivers an aggregate amount of ibogaine, ibogaine derivative, or pharmaceutically acceptable salt or solvate thereof of about 50 ng to about 1 μg per kg body weight per day. In some aspects, the composition is administered once per day. In some aspects, the composition is administered two or more times per day. In some embodiments, the composition is administered less than once a day, for example once every two days, once every three days, once every four days, once a week, etc.

In one embodiment, the average serum concentration of ibogaine, ibogaine derivative, or pharmaceutically acceptable salt or solvate thereof is from about 50 ng/mL to about 800 ng/mL or about 60 ng/mL to about 800 ng/mL. In one embodiment, the average serum concentration of the compound is from about 50 ng/mL to about 700 ng/mL or about 60 ng/mL to about 700 ng/mL. In one embodiment, the average serum concentration of the compound is from about 50 ng/mL to about 600 ng/mL, or about 60 ng/mL to about 600 ng/mL. In a preferred embodiment, the average serum concentration of the compound is from about 50 ng/mL to about 500 ng/mL, or about 60 ng/mL to about 500 ng/mL. In one embodiment, the average serum concentration of the compound is from about 50 ng/mL to about 400 ng/mL, or about 60 ng/mL to about 400 ng/mL. In one embodiment, the average serum concentration of the compound is from about 50 ng/mL to about 300 ng/mL, or about 60 ng/mL to about 300 ng/mL. In one embodiment, the average serum concentration of the compound is from about 50 ng/mL to about 200 ng/mL, or about 60 ng/mL to about 200 ng/mL. In one embodiment, the average serum concentration of the compound is from about 50 ng/mL to about 100 ng/mL, or about 60 ng/mL to about 100 ng/mL. The ranges include both extremes as well as any subranges between.

In one embodiment, the dosage or aggregate dosage of ibogaine, ibogaine derivative, or pharmaceutically acceptable salt or solvate thereof is from greater than about 1 mg/kg to about 8 mg/kg body weight per day. The aggregate dosage is the combined dosage, for example the total amount of ibogaine, ibogaine derivative, or pharmaceutically acceptable salt or solvate thereof administered over a 24-hour period where smaller amounts are administered more than once per day. In one embodiment, the dosage or aggregate dosage of ibogaine, ibogaine derivative, or pharmaceutically acceptable salt or solvate thereof is from about 1.3 mg/kg to about 7 mg/kg body weight. In one embodiment, the dosage or aggregate dosage of ibogaine, ibogaine derivative, or pharmaceutically acceptable salt or solvate thereof is from about 1.3 mg/kg to about 6 mg/kg body weight. In one embodiment, the dosage or aggregate dosage of ibogaine, ibogaine derivative, or pharmaceutically acceptable salt or solvate thereof is from about 1.3 mg/kg to about 5 mg/kg body weight. In a preferred embodiment, the dosage or aggregate dosage of ibogaine, ibogaine derivative, or pharmaceutically acceptable salt or solvate thereof is from about 1.3 mg/kg to about 4 mg/kg body weight. In one embodiment, the dosage or aggregate dosage of ibogaine, ibogaine derivative, or pharmaceutically acceptable salt or solvate thereof is from about 1.3 mg/kg to about 3 mg/kg body weight. In one embodiment, the dosage or aggregate dosage of ibogaine, ibogaine derivative, or pharmaceutically acceptable salt or solvate thereof is from about 1.3 mg/kg to about 2 mg/kg body weight. In one embodiment, the dosage or aggregate dosage of ibogaine, ibogaine derivative, or pharmaceutically acceptable salt or solvate thereof is from about 1.5 mg/kg to about 3 mg/kg body weight. In one embodiment, the dosage or aggregate dosage of ibogaine, ibogaine derivative, or pharmaceutically acceptable salt or solvate thereof is from about 1.7 mg/kg to about 3 mg/kg body weight. In one embodiment, the dosage or aggregate dosage of ibogaine, ibogaine derivative, or pharmaceutically acceptable salt or solvate thereof is from about 2 mg/kg to about 4 mg/kg body weight. In one embodiment, the dosage or aggregate dosage of ibogaine, ibogaine derivative, or pharmaceutically acceptable salt or solvate thereof is from about 2 mg/kg to about 3 mg/kg body weight. In one embodiment, the dosage or aggregate dosage of ibogaine, ibogaine derivative, or pharmaceutically acceptable salt or solvate thereof is about 2 mg/kg body weight. The ranges include both extremes as well as any subranges there between.

In one embodiment, the dosage or aggregate dosage of ibogaine, ibogaine derivative, or pharmaceutically acceptable salt or solvate thereof is about 8 mg/kg body weight per day. In one embodiment, the dosage or aggregate dosage of ibogaine, ibogaine derivative, or pharmaceutically acceptable salt or solvate thereof is about 7 mg/kg body weight per day. In one embodiment, the dosage or aggregate dosage of ibogaine, ibogaine derivative, or pharmaceutically acceptable salt or solvate thereof is about 6 mg/kg body weight per day. In one embodiment, the dosage or aggregate dosage of ibogaine, ibogaine derivative, or pharmaceutically acceptable salt or solvate thereof is about 5 mg/kg body weight per day. In one embodiment, the dosage or aggregate dosage of ibogaine, ibogaine derivative, or pharmaceutically acceptable salt or solvate thereof is about 4 mg/kg body weight per day. In one embodiment, the dosage or aggregate dosage of ibogaine, ibogaine derivative, or pharmaceutically acceptable salt or solvate thereof is about 3 mg/kg body weight per day. In one embodiment, the dosage or aggregate dosage of ibogaine, ibogaine derivative, or pharmaceutically acceptable salt or solvate thereof is about 2 mg/kg body weight per day. In one embodiment, the dosage or aggregate dosage of ibogaine, ibogaine derivative, or pharmaceutically acceptable salt or solvate thereof is about 1.7 mg/kg body weight per day. In one embodiment, the dosage or aggregate dosage of ibogaine, ibogaine derivative, or pharmaceutically acceptable salt or solvate thereof is about 1.5 mg/kg body weight per day. In one embodiment, the dosage or aggregate dosage of ibogaine, ibogaine derivative, or pharmaceutically acceptable salt or solvate thereof is about 1.3 mg/kg body weight per day. In one embodiment, the dosage or aggregate dosage of ibogaine, ibogaine derivative, or pharmaceutically acceptable salt or solvate thereof is about 1 mg/kg body weight per day.

In one embodiment, the dosage or aggregate dosage of compound is from about 1 mg to about 4 mg per kg body weight per day. The aggregate dosage is the combined dosage, for example the total amount of ibogaine, ibogaine derivative, or pharmaceutically acceptable salt or solvate thereof administered over a 24-hour period where smaller amounts are administered more than once per day.

In one embodiment, the dosage or aggregate dosage of ibogaine, ibogaine derivative, or pharmaceutically acceptable salt or solvate thereof is between about 70 mg and about 150 mg. In one embodiment, the dosage or aggregate dosage of ibogaine, ibogaine derivative, or pharmaceutically acceptable salt or solvate thereof is between about 75 mg and about 150 mg. In one embodiment, the dosage or aggregate dosage of ibogaine, ibogaine derivative, or pharmaceutically acceptable salt or solvate thereof is between about 80 mg and about 140 mg. In one embodiment, the dosage or aggregate dosage of ibogaine, ibogaine derivative, or pharmaceutically acceptable salt or solvate thereof is between about 90 mg and about 140 mg. In one embodiment, the dosage or aggregate dosage of ibogaine, ibogaine derivative, or pharmaceutically acceptable salt or solvate thereof is between about 90 mg and about 130 mg. In one embodiment, the dosage or aggregate dosage of ibogaine, ibogaine derivative, or pharmaceutically acceptable salt or solvate thereof is between about 100 mg and about 130 mg. In one embodiment, the dosage or aggregate dosage of ibogaine, ibogaine derivative, or pharmaceutically acceptable salt or solvate thereof is between about 110 mg and about 130 mg. The ranges include both extremes as well as any subrange or subvalue there between.

In one embodiment, the average serum concentration of ibogaine, ibogaine derivative, or pharmaceutically acceptable salt or solvate thereof is from about 50 ng/mL to about 800 ng/mL or about 60 ng/mL to about 800 ng/mL. In one embodiment, the average serum concentration of ibogaine, ibogaine derivative, or pharmaceutically acceptable salt or solvate thereof is from about 50 ng/mL to about 700 ng/mL or about 60 ng/mL to about 700 ng/mL. In one embodiment, the average serum concentration of ibogaine, ibogaine derivative, or pharmaceutically acceptable salt or solvate thereof is from about 50 ng/mL to about 600 ng/mL, or about 60 ng/mL to about 600 ng/mL. In a preferred embodiment, the average serum concentration of ibogaine, ibogaine derivative, or pharmaceutically acceptable salt or solvate thereof is from about 50 ng/mL to about 500 ng/mL, or about 60 ng/mL to about 500 ng/mL. In one embodiment, the average serum concentration of ibogaine, ibogaine derivative, or pharmaceutically acceptable salt or solvate thereof is from about 50 ng/mL to about 400 ng/mL, or about 60 ng/mL to about 400 ng/mL. In one embodiment, the average serum concentration of ibogaine, ibogaine derivative, or pharmaceutically acceptable salt or solvate thereof is from about 50 ng/mL to about 300 ng/mL, or about 60 ng/mL to about 300 ng/mL. In one embodiment, the average serum concentration of ibogaine, ibogaine derivative, or pharmaceutically acceptable salt or solvate thereof is from about 50 ng/mL to about 200 ng/mL, or about 60 ng/mL to about 200 ng/mL. In one embodiment, the average serum concentration of ibogaine, ibogaine derivative, or pharmaceutically acceptable salt or solvate thereof is from about 50 ng/mL to about 100 ng/mL, or about 60 ng/mL to about 100 ng/mL. The ranges include both extremes as well as any subranges between.

In one embodiment, the average serum concentration of ibogaine, ibogaine derivative, or pharmaceutically acceptable salt or solvate thereof is from about 50 ng/mL to about 180 ng/mL, or about 60 ng/mL to about 180 ng/mL. In one embodiment, the average serum concentration of ibogaine, ibogaine derivative, or pharmaceutically acceptable salt or solvate thereof is from about 50 ng/mL to about 150 ng/mL, or about 60 ng/mL to about 150 ng/mL. In one embodiment, the average serum concentration of ibogaine, ibogaine derivative, or pharmaceutically acceptable salt or solvate thereof is from about 50 ng/mL to about 100 ng/mL, or about 60 ng/mL to about 100 ng/mL. In one embodiment, the average serum concentration of ibogaine, ibogaine derivative, or pharmaceutically acceptable salt or solvate thereof is from about 80 ng/mL to about 150 ng/mL. In one embodiment, the average serum concentration of ibogaine, ibogaine derivative, or pharmaceutically acceptable salt or solvate thereof is from about 80 ng/mL to about 100 ng/mL. In one embodiment, such a dosing regimen provides an average serum concentration of ibogaine, ibogaine derivative, or pharmaceutically acceptable salt or solvate thereof of about 50 ng/mL to about 180 ng/mL. In one embodiment, the one or more additional doses maintain an average serum concentration of about 50 ng/mL to about 180 ng/mL over a period of time. The ranges include both extremes as well as any subrange or subvalue there between.

In one embodiment, the dosage of ibogaine, ibogaine derivative, or pharmaceutically acceptable salt or solvate thereof provides a serum concentration of between about 1000 ng*hr/mL and about 6000 ng*hr/mL. In one embodiment, the dosage of ibogaine, ibogaine derivative, or pharmaceutically acceptable salt or solvate thereof provides a serum concentration of between about 1200 ng*hr/mL and about 5800 ng*hr/mL. In one embodiment, the dosage of ibogaine, ibogaine derivative, or pharmaceutically acceptable salt or solvate thereof provides a serum concentration of between about 1200 ng*hr/mL and about 5500 ng*hr/mL. The ranges include both extremes as well as any subrange or subvalue there between.

In one embodiment, the dosage of ibogaine, ibogaine derivative, or pharmaceutically acceptable salt or solvate thereof provides a maximum serum concentration (Cmax) of less than about 250 ng/mL. In one embodiment, the dosage of ibogaine, ibogaine derivative, or pharmaceutically acceptable salt or solvate thereof provides a Cmax between about 40 ng/mL and about 250 ng/mL. In a preferred embodiment, the dosage of ibogaine, ibogaine derivative, or pharmaceutically acceptable salt or solvate thereof provides a Cmax between about 60 ng/mL and about 200 ng/mL. In one embodiment, the dosage of ibogaine, ibogaine derivative, or pharmaceutically acceptable salt or solvate thereof provides a Cmax between about 100 ng/mL and about 180 ng/mL. The ranges include both extremes as well as any subrange or subvalue there between.

In another embodiment, there is provided a unit dose of ibogaine, ibogaine derivative, or pharmaceutically acceptable salt or solvate thereof which is about 50 mg to about 200 mg per dose. In one embodiment, the unit dose is about 50 to about 120 mg per dose. In one embodiment, the unit dose is about 120 mg per dose. It being understood that the term “unit dose” means a dose sufficient to provide therapeutic results whether given all at once or serially over a period of time.

In some embodiments, the patient is administered an initial dose of ibogaine, ibogaine derivative, or pharmaceutically acceptable salt or solvate thereof followed by one or more additional doses.

In some embodiments, the initial dose of ibogaine, ibogaine derivative, or pharmaceutically acceptable salt or solvate thereof is from about 75 mg to about 120 mg. In one embodiment, the initial dose is about 75 mg. In one embodiment, the initial dose is about 80 mg. In one embodiment, the initial dose is about 85 mg. In one embodiment, the initial dose is about 90 mg. In one embodiment, the initial dose is about 95 mg. In one embodiment, the initial dose is about 100 mg. In one embodiment, the initial dose is about 105 mg. In one embodiment, the initial dose is about 110 mg. In one embodiment, the initial dose is about 115 mg. In one embodiment, the initial dose is about 120 mg.

In some embodiments, the one or more additional doses are lower than the initial dose. In one embodiment, the one or more additional doses are from about 5 mg to about 50 mg. In one embodiment, the one or more additional doses may or may not comprise the same amount of ibogaine, ibogaine derivative, or pharmaceutically acceptable salt or solvate thereof. In one embodiment, at least one additional dose is about 5 mg. In one embodiment, at least one additional dose is about 10 mg. In one embodiment, at least one additional dose is about 15 mg. In one embodiment, at least one additional dose is about 20 mg. In one embodiment, at least one additional dose is about 25 mg. In one embodiment, at least one additional dose is about 30 mg. In one embodiment, at least one additional dose is about 35 mg. In one embodiment, at least one additional dose is about 40 mg. In one embodiment, at least one additional dose is about 45 mg. In one embodiment, at least one additional dose is about 50 mg.

In one embodiment, the one or more additional doses are administered periodically. In one embodiment, the one or more additional doses are administered approximately every 4 hours. In one embodiment, the one or more additional doses are administered every 6 hours. In one embodiment, the one or more additional doses are administered approximately every 8 hours. In one embodiment, the one or more additional doses are administered approximately every 10 hours. In one embodiment, the one or more additional doses are administered approximately every 12 hours. In one embodiment, the one or more additional doses are administered approximately every 18 hours. In one embodiment, the one or more additional doses are administered approximately every 24 hours. In one embodiment, the one or more additional doses are administered approximately every 36 hours. In one embodiment, the one or more additional doses are administered approximately every 48 hours.

In some embodiments, the patient is administered a high (therapeutic) dose of ibogaine, ibogaine derivative, or pharmaceutically acceptable salt or solvate thereof for a period of time to ameliorate the most significant symptoms of a disease or disorder, and then is administered a lower (maintenance) dose to prevent relapse. In some embodiments, the patient is administered a therapeutic dose of ibogaine, ibogaine derivative, or pharmaceutically acceptable salt or solvate thereof for a period of time to ameliorate the most significant symptoms, and then is administered a decreasing (tapered) amount of ibogaine, ibogaine derivative, or pharmaceutically acceptable salt or solvate thereof over time until the maintenance dose is reached.

In one embodiment, ibogaine is administered at an amount by weight that is twice that administered for noribogaine for treating a same or similar condition. For example, and without limitation, an administration of a dose 80 mg ibogaine approximates a dose of 40 mg noribogaine.

### Maintenance Dose

In some embodiments, the maintenance dose of ibogaine, ibogaine derivative, or pharmaceutically acceptable salt or solvate thereof is about 10% to about 80% of the therapeutic dose. In some embodiments, the maintenance dose of ibogaine, ibogaine derivative, or pharmaceutically acceptable salt or solvate thereof is about 70% of the therapeutic dose. In some embodiments, the maintenance dose is about 60% of the therapeutic dose. In some embodiments, the maintenance dose is about 50% of the therapeutic dose. In some embodiments, the maintenance dose is about 40% of the therapeutic dose. In some embodiments, the maintenance dose is about 30% of the therapeutic dose. In some embodiments, the maintenance dose is about 20% of the therapeutic dose. In some embodiments, the maintenance dose is about 10% of the therapeutic dose.

In some embodiments, the maintenance average serum level of ibogaine, ibogaine derivative, or pharmaceutically acceptable salt or solvate thereof is about 10% to about 80% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 70% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 60% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 50% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 40% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 30% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 20% of the therapeutic average serum level of ibogaine, ibogaine derivative, or pharmaceutically acceptable salt or solvate thereof. In some embodiments, the maintenance average serum level of ibogaine, ibogaine derivative, or pharmaceutically acceptable salt or solvate thereof is about 10% of the therapeutic average serum level of ibogaine, ibogaine derivative, or pharmaceutically acceptable salt or solvate thereof.

### Tapered Dosing

In some embodiments, the therapeutic dose of ibogaine, ibogaine derivative, or pharmaceutically acceptable salt or solvate thereof is a tapered dosing over a period of time, during which the patient is detoxified, for example, without suffering significant acute withdrawal symptoms. Without being bound by theory, it is believed that tapering will allow the full therapeutic effect of the compound with less prolongation of the QT interval. Tapering involves administration of one or more subsequently lower doses of the compound over time. For example, in some embodiments, the first tapered dose is about 50% to about 95% of the first or original dose. In some embodiments, the second tapered dose is about 40% to about 90% of the first or original dose. In some embodiments, the third tapered dose is about 30% to about 85% of the first or original dose. In some embodiments, the fourth tapered dose is about 20% to about 80% of the first or original dose. In some embodiments, the fifth tapered dose is about 10% to about 75% of the first or original dose.

In some embodiments, the first tapered dose is given after the first dose of ibogaine, ibogaine derivative, or pharmaceutically acceptable salt or solvate thereof. In some embodiments, the first tapered dose is given after the second, third, or a subsequent dose of compound. The first tapered dose may be administered at any time after the previous dose of compound. The first tapered dose can be given once, for example, followed by subsequent further tapered doses, or it can be given multiple times with or without subsequent, further tapered doses (e.g., second, third, fourth, etc. tapered doses), which likewise can be given once or over multiple administrations, for example. In some embodiments, the first tapered dose is administered one hour, 6 hours, 12 hours, 18 hours, 24 hours, 36 hours, 48 hours, or more after the previous dose of compound. Similarly. second, third, fourth, etc. tapered doses, if given, can be given one hour, 6 hours, 12 hours, 18 hours, 24 hours, 36 hours, 48 hours, or more after the previous dose of compound.

In some embodiments, one tapered dose is given to achieve the desired lower therapeutic dose. In some embodiments, two tapered doses are given to achieve the desired lower therapeutic dose. In some embodiments, three tapered doses are given to achieve the desired lower therapeutic dose. In some embodiments, four or more tapered doses are given to achieve the desired lower therapeutic dose. Determination of the tapered doses, number of tapered doses, and the like can be readily made a qualified clinician.

In one embodiment, the QT interval is not prolonged more than about 50 ms. In one embodiment, the QT interval is not prolonged more than about 40 ms. In one embodiment, the QT interval is not prolonged more than about 30 ms. In one embodiment, the QT interval is not prolonged more than about 20 ms. In one embodiment, prolongation of the QT interval is equivalent to or less than the prolongation observed for methadone-treated patients.

In some embodiments, the patient is administered periodically, such as once, twice, three time, four times or five time daily with ibogaine, ibogaine derivative, or pharmaceutically acceptable salt or solvate thereof. In some embodiments, the administration is once daily, or once every second day, once every third day, three times a week, twice a week, or once a week. The dosage and frequency of the administration depends on the route of administration, dosage, age and body weight of the patient, condition of the patient, without limitation. Determination of dosage and frequency suitable for the present technology can be readily made a qualified clinician.

A ibogaine, ibogaine derivative, or pharmaceutically acceptable salt or solvate thereof suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intra-arterial, intramuscular, intraperitoneal, intracutaneous and subcutaneous routes may also be used. Possible dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A. Oslo editor, Easton Pa. 1980).

In a preferred embodiment, ibogaine, ibogaine derivative, or pharmaceutically acceptable salt or solvate thereof is administered orally, which may conveniently be provided in tablet, caplet, sublingual, liquid or capsule form. In certain embodiments, the compound is provided as a pharmaceutically acceptable salt, for example ibogaine HCl, with dosages reported as the amount of free base compound. In some embodiments, the pharmaceutically acceptable salt is provided in hard gelatin capsules containing only the salt with no excipients.

The patient may suffer from addiction to any opioid or opiate or opioid-like drug. In a preferred embodiment, the opioid or opioid-like drug is selected from the group consisting of heroin, cocaine, opiate, methadone, morphine, codeine, oxycodone, hydrocodone, and methamphetamine. In one embodiment, the opioid or opioid-like drug is heroin. In one embodiment, the opioid or opioid-like drug is methadone. In one embodiment, the opioid or opioid-like drug is morphine. In one embodiment, the opioid or opioid-like drug is oxycodone. In one embodiment, the opioid or opioid-like drug is hydrocodone.

### Maintenance Administration

In one aspect, this invention relates to treatment or attenuation of post-acute withdrawal from opioids or opioid-like drug in an addicted patient with a maintenance amount of ibogaine, ibogaine derivative, or pharmaceutically acceptable salt or solvate thereof.

In some aspects, this invention relates to a method to prevent relapse of opioid or opioid-like drug abuse in an addicted patient treated to ameliorate said abuse, said method comprising periodically administering to said patient a maintenance dosage of ibogaine, ibogaine derivative, or pharmaceutically acceptable salt or solvate thereof.

In some embodiments, the patient undergoes long-term (e.g., one year or longer) treatment with maintenance doses of ibogaine, ibogaine derivative, or pharmaceutically acceptable salt or solvate thereof. In some embodiments, the patient is treated for acute withdrawal with therapeutic doses of ibogaine, ibogaine derivative, or pharmaceutically acceptable salt or solvate thereof as described above, and then the amount of compound is reduced to maintenance levels after acute withdrawal symptoms would be expected to have subsided. Acute withdrawal symptoms generally are the most pronounced in the first 48 to 72 hours after cessation of the drug of addiction, although acute withdrawal may last as long as a week or more.

In some embodiments, the patient is administered a high (therapeutic) dose of ibogaine, ibogaine derivative, or pharmaceutically acceptable salt or solvate thereof for a period of time to ameliorate the most significant withdraw symptoms, and then is administered a lower (maintenance) dose to prevent relapse to opioid or opioid-like drug use. In some embodiments, the patient is administered a therapeutic dose of ibogaine, ibogaine derivative, or pharmaceutically acceptable salt or solvate thereof for a period of time to ameliorate the most significant withdraw symptoms, and then is administered a decreasing (tapered) amount of the compound or pharmaceutically acceptable salt thereof over time until the maintenance dose is reached.

In some embodiments, the maintenance dose of ibogaine, ibogaine derivative, or a pharmaceutically acceptable salt thereof is 70% of the therapeutic dose. In some embodiments, the maintenance dose is 60% of the therapeutic dose. In some embodiments, the maintenance dose is 50% of the therapeutic dose. In some embodiments, the maintenance dose is 40% of the therapeutic dose. In some embodiments, the maintenance dose is 30% of the therapeutic dose. In some embodiments, the maintenance dose is 20% of the therapeutic dose. In some embodiments, the maintenance dose is 10% of the therapeutic dose

In some embodiments, the maintenance average serum level of compound is 70% of the therapeutic average serum level. In some embodiments, the maintenance average serum level of compound is 60% of the therapeutic average serum level. In some embodiments, the maintenance average serum level of compound is 50% of the therapeutic average serum level. In some embodiments, the maintenance average serum level of compound is 40% of the therapeutic average serum level. In some embodiments, the maintenance average serum level of compound is 30% of the therapeutic average serum level. In some embodiments, the maintenance average serum level of compound is 20% of the therapeutic average serum level. In some embodiments, the maintenance average serum level of compound is 10% of the therapeutic average serum level.

In one embodiment, the therapeutic dose is tapered over time until the desired maintenance dose is reached. For example, in some embodiments, the first tapered dose is about 50% to about 95% of the therapeutic dose. In some embodiments, the second tapered dose is about 40% to about 90% of the therapeutic dose. In some embodiments, the third tapered dose is about 30% to about 85% of the therapeutic dose. In some embodiments, the fourth tapered dose is about 20% to about 80% of the therapeutic dose. In some embodiments, the fifth tapered dose is about 10% to about 75% of the therapeutic dose. In some embodiments, one tapered dose is given to achieve the maintenance dose. In some embodiments, two tapered doses are given to achieve the maintenance dose. In some embodiments, three tapered doses are given to achieve the maintenance dose. In some embodiments, four or more tapered doses are given to achieve the maintenance dose. Determination of the tapered doses, number of tapered doses, and the like can be readily made a qualified clinician.

In one embodiment, the QT interval is not prolonged more than 30 ms. In a preferred embodiment, the QT interval is not prolonged more than 20 ms.

In some embodiments, the patient is administered periodically, such as once. twice, three time, four times or five time daily with ibogaine, ibogaine derivative, or pharmaceutically acceptable salt or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

Ibogaine, ibogaine derivative, or pharmaceutically acceptable salt or solvate thereof suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intra-arterial, intramuscular, intraperitoneal, intracutaneous and subcutaneous routes may also be used. Possible dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences. 16th ed., A. Oslo editor. Easton Pa. 1980).

Ibogaine or an ibogaine derivative can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing ibogaine may be prepared using conventional techniques that may include sterile isotonic saline, water, 1,3-butanediol, ethanol, 1,2-propylene glycol, polyglycols mixed with water, Ringer's solution, etc.

The compositions utilized herein may be formulated for aerosol administration, particularly to the respiratory tract and including intrapulmonary or intranasal administration. The compound will generally have a small particle size, for example of the order of 5 microns or less. Such a particle size may be obtained by means known in the art, for example by micronization. The active ingredient may be provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidine. In some embodiments, the powder carrier will form a gel in the nasal cavity. The powder composition may be presented in unit dose form, for example in capsules or cartridges, gelatin or blister packs, from which the powder may be administered by means of an inhaler.

In a preferred embodiment, ibogaine, ibogaine derivative, or pharmaceutically acceptable salt or solvate thereof is administered orally, which may conveniently be provided in tablet, caplet, sublingual, liquid or capsule form. In certain embodiments, the compound is provided as a pharmaceutically acceptable salt, for example ibogaine HCl, with dosages reported as the amount of free base compound. In some embodiments, the pharmaceutically acceptable salt is provided in hard gelatin capsules containing only the salt with no excipients.

The patient may suffer from addiction to any opioid or opiate, or opioid-like drug. In a preferred embodiment, the opioid or opioid-like drug is selected from the group consisting of heroin, cocaine, opiate, methadone, morphine, codeine, hydrocodone, oxycodone, and methamphetamine. In one embodiment, the opioid or opioid-like drug is heroin. In one embodiment, the opioid or opioid-like drug is methadone. In one embodiment, the opioid or opioid-like drug is morphine. In one embodiment, the opioid or opioid-like drug is hydrocodone. In one embodiment, the opioid or opioid-like drug is oxycodone.

### Patient Pre-Screening and Monitoring

Pre-screening of patients before treatment with ibogaine, ibogaine derivative, or pharmaceutically acceptable salt or solvate thereof and/or monitoring of patients during treatment may be required to ensure that QT interval is not prolonged beyond a certain value. For example, QT interval greater than 500 ms can be considered dangerous for individual patients. Pre-screening and/or monitoring may be necessary at high dosage levels.

In a preferred embodiment, a patient receiving a therapeutic dose of ibogaine, ibogaine derivative, or pharmaceutically acceptable salt or solvate thereof is monitored in a clinical setting. Monitoring may be necessary to ensure the QT interval is not prolonged to an unacceptable degree. A “clinical setting” refers to an inpatient setting (e.g., inpatient clinic, hospital, rehabilitation facility) or an outpatient setting with frequent, regular monitoring (e.g., outpatient clinic that is visited daily to receive dose and monitoring). Monitoring includes monitoring of QT interval. Methods for monitoring of QT interval are well-known in the art, for example by ECG.

In one embodiment, a patient receiving a maintenance dose of ibogaine, ibogaine derivative, or pharmaceutically acceptable salt or solvate thereof is not monitored in a clinical setting. In one embodiment, a patient receiving a maintenance dose of ibogaine, ibogaine derivative, or pharmaceutically acceptable salt or solvate thereof is monitored periodically, for example daily, weekly, monthly, or occasionally.

In one aspect, this invention relates to a method for treating opioid or opioid-like drug abuse and/or symptoms of withdrawal in an addicted patient, comprising selecting an opioid-or opioid-like drug-addicted patient who is prescreened to evaluate the patient's expected tolerance for prolongation of QT interval, administering to the patient a dosage of ibogaine, ibogaine derivative, or pharmaceutically acceptable salt or solvate thereof that provides an average serum concentration of about 50 ng/mL to about 850 ng/mL, said concentration being sufficient to inhibit or ameliorate said abuse or symptoms while maintaining a QT interval of less than 500 ms during said treatment. In some embodiments, the concentration is sufficient to attenuate said abuse or symptoms while maintaining a QT interval of less than about 470 ms during treatment. Preferably, the concentration is sufficient to attenuate said abuse or symptoms while maintaining a QT interval of less than about 450 ms during treatment. In one embodiment, the concentration is sufficient to attenuate said abuse or symptoms while maintaining a QT interval of less than about 420 ms during treatment.

In one embodiment, prescreening of the patient comprises ascertaining that treatment with ibogaine, ibogaine derivative, or pharmaceutically acceptable salt or solvate thereof will not result in a QT interval over about 500 ms. In one embodiment, prescreening of the patient comprises ascertaining that treatment with ibogaine, ibogaine derivative, or pharmaceutically acceptable salt or solvate thereof will not result in a QT interval over about 470 ms. In one embodiment, prescreening comprises ascertaining that treatment with ibogaine, ibogaine derivative, or pharmaceutically acceptable salt or solvate thereof will not result in a QT interval over about 450 ms. In one embodiment, prescreening comprises ascertaining that treatment with ibogaine, ibogaine derivative, or pharmaceutically acceptable salt or solvate thereof will not result in a QT interval over about 420 ms. In one embodiment, prescreening comprises determining the patient's pre-treatment QT interval.

As it relates to pre-screening or pre-selection of patients, patients may be selected based on any criteria as determined by the skilled clinician. Such criteria may include, by way of non-limiting example, pre-treatment QT interval, pre-existing cardiac conditions, risk of cardiac conditions, age, sex, general health, and the like. The following are examples of selection criteria for disallowing treatment or restricting dose of ibogaine, ibogaine derivative, or pharmaceutically acceptable salt or solvate thereof administered to the patient: high QT interval before treatment (e.g., such that there is a risk of the patient's QT interval exceeding 500 ms during treatment); congenital long QT syndrome; bradycardia; hypokalemia or hypomagnesaemia; recent acute myocardial infarction; uncompensated heart failure; and taking other drugs that increase QT interval. In some embodiments, the methods can include selecting and/or administering/providing ibogaine, ibogaine derivative, or pharmaceutically acceptable salt or solvate thereof to a patient that lacks one more of such criteria.

In one embodiment, this invention relates to pre-screening a patient to determine if the patient is at risk for prolongation of the QT interval beyond a safe level. In one embodiment, a patient at risk for prolongation of the QT interval beyond a safe level is not administered ibogaine, ibogaine derivative, or pharmaceutically acceptable salt or solvate thereof. In one embodiment, a patient at risk for prolongation of the QT interval beyond a safe level is administered ibogaine, ibogaine derivative, or pharmaceutically acceptable salt or solvate thereof at a limited dosage.

In one embodiment, this invention relates to monitoring a patient who is administered a therapeutic dose of ibogaine, ibogaine derivative, or pharmaceutically acceptable salt or solvate thereof. In one embodiment, the dose is reduced if the patient has serious adverse side effects. In one embodiment, treatment is discontinued if the patient has serious adverse side effects. In one embodiment, the adverse side effect is a QT interval that is prolonged beyond a safe level. The determination of a safe level of prolongation is within the skill of a qualified clinician.

### Kit of Parts

One aspect of this invention is directed to a kit of parts for the treatment of opioid or opioid-like drug abuse and/or symptoms of withdrawal in an addicted patient, wherein the kit comprises a composition comprising ibogaine, ibogaine derivative, or pharmaceutically acceptable salt or solvate thereof and a means for administering the composition to a patient in need thereof. The means for administration to a patient can include, for example, any one or combination of ibogaine, ibogaine derivative, or pharmaceutically acceptable salt or solvate thereof a transdermal patch, a syringe, a needle, an IV bag comprising the composition, a vial comprising the composition, an inhaler comprising the composition, etc. In one embodiment, the kit of parts further comprises instructions for dosing and/or administration of the composition.

In some aspects, the invention is directed to a kit of parts for administration of ibogaine, ibogaine derivative, or pharmaceutically acceptable salt or solvate thereof the kit comprising multiple delivery vehicles, wherein each delivery vehicle contains a discrete amount of compound and further wherein each delivery vehicle is identified by the amount of compound provided therein; and optionally further comprising a dosing treatment schedule in a readable medium. In some embodiments, the dosing treatment schedule includes the amount of compound required to achieve each average serum level is provided. In some embodiments, the kit of parts includes a dosing treatment schedule that provides an attending clinician the ability to select a dosing regimen based on the sex of the patient, mass of the patient, compound, and the serum level that the clinician desires to achieve. In some embodiments, the dosing treatment schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “delivery vehicle” as used herein refers to any formulation that can be used for administration of ibogaine, ibogaine derivative, or pharmaceutically acceptable salt or solvate thereof to a patient. Non-limiting, exemplary delivery vehicles include caplets, pills, capsules, tablets, powder, liquid, or any other form by which the drug can be administered. Delivery vehicles may be intended for administration by oral, inhaled, injected, or any other means.

The term “readable medium” as used herein refers to a representation of data that can be read, for example, by a human or by a machine Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some aspects, the machine-readable medium comprises software that contains information regarding dosing schedules for the unit dose form of ibogaine, ibogaine derivative, or pharmaceutically acceptable salt or solvate thereof and optionally other drug information. In some embodiments, the software may be interactive, such that the attending clinician or other medical professional can enter patient information. In a non-limiting example, the medical professional may enter the weight and sex of the patient to be treated, and the software program provides a recommended dosing regimen based on the information entered. The amount and timing of compound recommended to be delivered will be within the dosages that result in the serum concentrations as provided herein.

In some embodiments, the kit of parts comprises multiple delivery vehicles in a variety of dosing options. For example, the kit of parts may comprise pills or tablets in multiple dosages, such as 240 mg, 120 mg, 90 mg, 60 mg, 30 mg, 20 mg, and/or 10 mg of ibogaine, ibogaine derivative, or pharmaceutically acceptable salt or solvate thereof per pill. Each pill is labeled such that the medical professional and/or patient can easily distinguish different dosages. Labeling may be based on printing or embossing on the pill, shape of the pill, color of pill, the location of the pill in a separate, labeled compartment within the kit, and/or any other distinguishing features of the pill. In some embodiments, all of the delivery vehicles within a kit are intended for one patient. In some embodiments, the delivery vehicles within a kit are intended for multiple patients.

One aspect of this invention is directed to a kit of parts for the treatment of opioid, or opioid-like drug, abuse and/or symptoms of withdrawal in an addicted patient, wherein the kit comprises a unit dose form of ibogaine, ibogaine derivative, or pharmaceutically acceptable salt or solvate thereof. The unit dose form provides a patient with an average serum level of compound of from about 50 ng/ml to about 800 ng/ml or about 60 ng/mL to about 800 ng/mL.

In some embodiments, the unit dose form comprises one or multiple dosages to be administered periodically, such as once, twice, three time, four times or five time daily with ibogaine, ibogaine derivative, or pharmaceutically acceptable salt or solvate thereof. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the addiction. Determination of the unit dose form providing a dosage and frequency suitable for a given patient can readily be made by a qualified clinician.

These dose ranges may be achieved by transdermal, oral, or parenteral administration of ibogaine, ibogaine derivative, or pharmaceutically acceptable salt or solvate thereof in unit dose form. Such unit dose form may conveniently be provided in transdermal patch, tablet, caplet, liquid or capsule form.

### Formulations

This invention further relates to pharmaceutically acceptable formulations comprising a unit dose of ibogaine, ibogaine derivative, or pharmaceutically acceptable salt or solvate thereof, wherein the amount of compound is sufficient to provide an average serum concentration of about 50 ng/mL to about 850 ng/mL when administered to a patient. In a preferred embodiment, the amount of compound is sufficient to provide an average serum concentration of about 50 ng/mL to about 400 ng/mL when administered to a patient.

In some embodiments, the unit dose of ibogaine, ibogaine derivative, or pharmaceutically acceptable salt or solvate thereof is administered in one or more dosings.

In one embodiment, the amount of ibogaine, ibogaine derivative, or pharmaceutically acceptable salt or solvate thereof is sufficient to provide an average serum concentration from about 50 ng/mL to about 800 ng/mL or about 60 ng/mL to about 800 ng/mL. In one embodiment, the amount of compound is sufficient to provide an average serum concentration from about 50 ng/mL to about 700 ng/mL or about 60 ng/mL to about 700 ng/mL. In one embodiment, the amount of compound is sufficient to provide an average serum concentration from about 50 ng/mL to about 600 ng/mL, or about 60 ng/mL to about 600 ng/mL. In a preferred embodiment, the amount of compound is sufficient to provide an average serum concentration from about 50 ng/mL to about 500 ng/mL, or about 60 ng/mL to about 500 ng/mL. In one embodiment, the amount of compound is sufficient to provide an average serum concentration from about 50 ng/ml to about 400 ng/mL, or about 60 ng/mL to about 400 ng/mL. In one embodiment, the amount of compound is sufficient to provide an average serum concentration from about 50 ng/mL to about 300 ng/mL, or about 60 ng/mL to about 300 ng/mL. In one embodiment, the amount of compound is sufficient to provide an average serum concentration from about 50 ng/mL to about 200 ng/mL, or about 60 ng/mL to about 200 ng/mL. In one embodiment, the amount of compound is sufficient to provide an average serum concentration from about 50 ng/mL to about 100 ng/mL, or about 60 ng/mL to about 100 ng/mL. The ranges include both extremes as well as any subranges between.

In some embodiments, the formulation is designed for periodic administration, such as once, twice, three time, four times or five time daily with ibogaine, ibogaine derivative, or pharmaceutically acceptable salt or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

In some embodiments, the formulation designed for administration in accordance with the methods provide herein can be suitable for a variety of delivery modes including, without limitation, oral and transdermal delivery. Formulations suitable for internal, pulmonary, rectal, nasal, vaginal, lingual, intravenous, intra-arterial, intramuscular, intraperitoneal, intracutaneous and subcutaneous routes may also be used. Possible formulations include tablets, capsules, pills, powders, aerosols, suppositories, parenterals. and oral liquids, including suspensions, solutions and emulsions. Sustained release dosage forms may also be used. All formulations may be prepared using methods that are standard in the art (see e.g., Remington's Pharmaceutical Sciences, 16th ed., A. Oslo editor, Easton Pa. 1980).

In a preferred embodiment, the formulation is designed for oral administration, which may conveniently be provided in tablet, caplet, sublingual, liquid or capsule form.

### EXAMPLES

The following Examples are intended to further illustrate certain embodiments of the disclosure and are not intended to limit its scope.

### Example 1. Efficacy of Ibogaine in Treating Opioid-Dependency in Humans

The efficacy of ibogaine is evaluated in opioid-dependent participants in a randomized, placebo-controlled, double-blind trial. Patients are administered 60 mg or 120 mg of the compound and QT interval is measured.

1.-19. (canceled)

20. A method for treating opioid or opioid-like drug abuse in a human patient addicted thereto, the method comprising intravenously administering a therapeutically effective amount of ibogaine or a pharmaceutically acceptable salt thereof to the patient, wherein the intravenous administration provides a serum concentration of about 50 ng/mL to about 850 ng/mL.

21. The method of claim 20, wherein the intravenous administration provides a serum ibogaine concentration of about 100 ng/mL to about 800 ng/mL.

22. The method of claim 20, wherein the intravenous administration provides a serum ibogaine concentration of about 100 ng/mL to about 700 ng/mL.

23. The method of claim 20, wherein the intravenous administration provides a serum ibogaine concentration of about 200 ng/mL to about 600 ng/mL.

24. The method of claim 20, wherein the intravenous administration attenuates withdrawal symptoms.

25. The method of claim 20, wherein the intravenous administration prevents relapse of opioid or opioid-like drug use.

26. The method of claim 20, wherein the opioid or opioid-like drug comprises fentanyl, hydrocodone, hydromorphone, morphine, oxycodone, buprenorphine, codeine, oripavine, thebaine, buprenorphine, methadone, meperidine, tramadol, tapentadol, levorphanol, oxymorphone, cocaine, ketamine, methamphetamine, opium, opium poppy, poppy straw, or an extract or concentrate thereof.

27. The method of claim 20, wherein the intravenous administration is once daily, once every second day, once every third day, three times a week, twice a week, or once a week.

28. A method for treating opioid or opioid-like drug abuse in a human patient addicted thereto, the method comprising intravenously administering about 1 mg/kg to about 8 mg/kg of ibogaine or a pharmaceutically acceptable salt thereof to the patient during a 24-hour period.

29. The method of claim 28, wherein about 1.3 mg/kg to about 8 mg/kg of ibogaine or a pharmaceutically acceptable salt thereof is intravenously administered to the patient during a 24-hour period.

30. The method of claim 28, wherein about 2 mg/kg to about 8 mg/kg of ibogaine or a pharmaceutically acceptable salt thereof is intravenously administered to the patient during a 24-hour period.

31. The method of claim 28, wherein about 2 mg/kg to about 7 mg/kg of ibogaine or a pharmaceutically acceptable salt thereof is intravenously administered to the patient during a 24-hour period.

32. The method of claim 28, wherein the intravenous administration attenuates withdrawal symptoms.

33. The method of claim 28, wherein the intravenous administration prevents relapse of opioid or opioid-like drug use.

34. The method of claim 28, wherein the opioid or opioid-like drug comprises fentanyl, hydrocodone, hydromorphone, morphine, oxycodone, buprenorphine, codeine, oripavine, thebaine, buprenorphine, methadone, meperidine, tramadol, tapentadol, levorphanol, oxymorphone, cocaine, ketamine, methamphetamine, opium, opium poppy, poppy straw, or an extract or concentrate thereof.

35. The method of claim 28, wherein the intravenous administration is once daily, once every second day, once every third day, three times a week, twice a week, or once a week.
